(19) United States
(12) Patent Application Publication
(10) Pub. No.: US 2024/0238268 A1
(43) Pub. Date: Jul. 18, 2024

(54) TREATMENTS AND METHODS FOR TREATING ALZHEIMER'S DISEASE

(71) Applicant: Board of Supervisors of Louisiana State University and Agricultural and Mechanical College, Baton Rouge, LA (US)

(72) Inventor: Hung Wen (Kevin) Lin, Shreveport, LA (US)

(73) Assignee: Board of Supervisors of Louisiana State University and Agricultural and Mechanical College, Baton Rouge, LA (US)

(21) Appl. No.: 18/690,468

(22) PCT Filed: Sep. 9, 2022
(86) PCT No.: PCT/US2022/043159

§ 371 Date: Mar. 8, 2024

Related U.S. Application Data
(60) Provisional application No. 63/242,146, filed on Sep. 9, 2021.

(51) Int. Cl. A61K 31/4545; A61K 38/45; A61P 25/28

(52) U.S. Cl. CPC A61K 31/4545; A61K 38/45; A61P 25/28

(57) ABSTRACT

Therapeutics and methods treating Alzheimer's disease or a pre-Alzheimer's disease condition in a patient comprising administering a pharmaceutical composition containing a therapeutically effective dose therapeutic, wherein the therapeutic contains a PRMT4 inhibitor, or a pharmaceutically acceptable salt, solvate, ester, amide, clathrate, stereoisomer, enantiomer, prodrug or analog thereof. According to a further embodiment, the PRMT4 inhibitor is one of TP-064 and SCF^{FBXO9}.

### CROSS REFERENCE TO RELATED APPLICATIONS/PRIORITY

The present invention claims priority to U.S. Provisional Patent Application No. 63/242,146 filed Sep. 9, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. 1R01NS096225-01A1 awarded by the National Institutes of Health. The government has certain rights in the invention.

### BACKGROUND

Alzheimer's disease (AD) is one of the most common and progressive genetic neurodegenerative disorders in the US with more than 6 million people currently living with AD, with risk factors often associated with age and sex. Women are more likely to develop a rapid progression of AD than men when predictive factors such as obesity, lifespan, and enhanced stroke severity are considered. Changes in the brain may begin a decade or more before symptoms appear. During this very early stage of AD, which may be considered pre symptomatic or pre-AD, toxic changes are taking place in the brain, including abnormal buildups of proteins that form amyloid plaques and tau tangles. Previously healthy neurons stop functioning, lose connections with other neurons, and die. Many other complex brain changes are thought to play a role in AD as well. However, despite the devastating impact that AD inflicts on millions of individuals, their families, and society at large, there is still no effective treatment for the disease known in the art. For the foregoing reasons, there is a pressing, but seemingly irresolvable need for a treatment of AD and pre-AD.

### SUMMARY

Wherefore, it is an object of the present invention to overcome the above mentioned shortcomings and drawbacks associated with the current technology. The present invention is directed to therapeutics and methods treating Alzheimer's disease or a pre-Alzheimer's disease condition in a patient comprising administering of a pharmaceutical compositions containing a therapeutically effective dose therapeutic, wherein the therapeutic contains a PRMT4 inhibitor, or a pharmaceutically acceptable salt, solvate, ester, amide, clathrate, stereoisomer, enantiomer, prodrug or analog thereof. According to a further embodiment, the PRMT4 inhibitor is one of TP-064 and SCF^{FBXO9}. According to a further embodiment, the PRMT4 inhibitor is TP-064. According to a further embodiment, the PRMT4 inhibitor is SCF^{FBXO9}. According to a further embodiment, the PRMT4 inhibitor is administered in a dosage of between 0.1 mg/kg and 10.0 mg/kg, with the dosage expressed in a ratio of mass of PRMT4 inhibitor to mass of patient. According to a further embodiment, the dosage is between 0.5 mg/kg and 5.0 mg/kg. According to a further embodiment, the dosage is between 1.0 mg/kg and 3.0 mg/kg. According to a further embodiment, the pharmaceutical composition is administered for at least 7 days. According to a further embodiment, the patient does not display memory loss symptomatic of Alzheimer's disease. According to a further embodiment, the patient displays memory loss symptomatic of Alzheimer's disease.

The present invention relates to pharmaceutical compositions of a therapeutic (e.g., a PRMT4 inhibitor), or a pharmaceutically acceptable salt, solvate, ester, amide, clathrate, stereoisomer, enantiomer, prodrug or analogs thereof, and use of these compositions for the treatment of AD or pre-AD.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is AD.

In certain embodiments, the AD or pre-AD is mild to moderate AD or pre-AD.

In further embodiments, the AD or pre-AD is moderate to severe AD.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of AD or pre-AD.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, EudragitR FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., a PRMT4 inhibitor, such as TP-064 or SCF^{FBXO9}, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., AD or pre-AD). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a schematic diagram of one embodiment of the disclosed method.

FIGS. 2A and 2B are capillary-based immunoassay and normalized graphs showing aged 3xTg-AD female mice have enhanced brain PRMT4 protein level. PRMT4 protein level was significantly increased in (FIG. 2A) cortex and (FIG. 2B) hippocampus of aged v. young 3xTg-AD female mice. PRMT4 protein (˜63 kDa) level was measured by capillary-based immunoassay and normalized to total protein. *p<0.05 v. young female 3xTg-AD mice, evaluated by unpaired t-test.

FIGS. 3A-3C show PRMT4 mRNA and protein level is enhanced in aged female 3xTg-AD brain. FIG. 3A is a graph showing PRMT4 and PRMT6 mRNA were significantly increased in the brain of aged 3xTg-AD female mice. mRNA for all known PRMT species (1-9) was assessed using RT-qPCR. FIGS. 3B and 3C are graphs and corresponding capillary-based immunoassays. FIG. 3B shows PRMT4 protein expression was significantly enhanced in aged female 3xTg-AD mice. FIG. 3C shows no change was observed in PRMT6 protein level in aged female 3xTg-AD mice compared to age- and sex-matched C57 mice. PRMT4 (˜63 kDa) and PRMT6 (˜42 kDa) protein level was measured by capillary-based immunoassay and normalized to total protein. * p<0.05 via one-way ANOVA with Tukey's post-hoc.

FIGS. 4A and 4B show PRMT4 protein is elevated in the blood of female ADRD patients and in AD brain. FIG. 4A shows relative PRMT4 protein level was significantly increased in the blood of ADRD female patients v. age-matched non-AD females. PRMT4 protein was quantified via capillary-phoresies and normalized to total protein. The number in parentheses indicate the number of patients. FIG. 4B shows representative images of post-mortem brain tissue of confirmed AD and non-AD patient was stained for PRMT4 (brown) and counterstained with hematoxylin (blue) showing higher expression of PRMT4 in AD brain. * p<0.05 v. non-AD females evaluated by one-way ANOVA with Tukey's post-hoc analysis.

FIGS. 5A-5D are capillary-based immunoassay and associated graphs showing PRMT4 protein level was significantly increased in the brain of aged 3xTg-AD female mice, but reduced with specific PRMT4 inhibitor (TP-064) or AAV knockdown of PRMT4. FIGS. 4A and 4B show PRMT4 protein level in cortex and hippocampus, respectively, was significantly increased in aged 3xTg-AD female mice (not males). FIGS. 4C and 4D show treatment with a specific PRMT4 inhibitor (TP-064, 30 mg/kg for 7 days, IP) reduced, respectively, cortical and hippocampal PRMT4 protein level in aged 3xTg-AD female mice. AAV knockdown of PRMT4 also resulted in reducedPRMT4 level. Relative PRMT4 protein level (˜63 kDa) was measured by capillary-based immunoassay and normalized to total protein. * p<0.05 v. aged male 3xTg-AD mice, #p<0.05 v. aged 3xTg-AD female mice evaluated by unpaired t-test for FIGS. 4A and 4B or one-way ANOVA with Tukey's post-hoc analyses for FIGS. 4C and 4D.

FIG. 6 is six immunofluorescence images showing PRMT4 is colocalized with NOTCH1 in cortical neurons of aged 3xTg-AD female mice. Representative immunofluorescence images of 3xTg-AD cortical neurons (NeuN, blue), showing PRMT4 (green), and NOTCH1 (red) colocalization near blood vessels (CD31, magenta). Images were obtained via confocal microscopy at 100× magnification.

FIGS. 7A and 7B show PRMT4 methylation of NOTCH1 is enhanced in aged 3xTg-AD female mice. In FIG. 7A, magnetic bead immunoprecipitation was performed for NOTCH1 in the whole-brain lysates of aged 3xTg-AD or C57 female mice. The precipitated NOTCH1 product was analyzed via capillary-based immunoassay for NOTCH1 (110 kDa) to verify purity, as well as asymmetric dimethylarginine (ADMA, 116 kDA) to determine the level of PRMT4 activity. In FIG. 7B, aged 3xTg-AD female mice have higher ADMA/NOTCH1 ratio v. age- and sex-matched C57. Since PRMT4 catalyzes the transfer of a methyl group to S-adenosyl-L-methionine to ADMA, the reaction is suggestive of PRMT4-mediated NOTCH1 methylation. * p<0.05 as compared to aged C57 evaluated by unpaired t-test.

FIG. 8 is a graphical representation of various experiments disclosed herein.

FIGS. 9A-9D are capillary-based immunoassay and associated graphs showing aged female 3xTg-AD mice have higher levels of brain amyloid precursor protein (APP) and phosphorylated Tau (pTau) v. aged C57 female mice. Relative protein levels of APP, shown in FIGS. 9A and 9B, and pTau, shown in 9C and 9D, in the cortex and hippocampus were measured by capillary-based immunoassay and normalized to total protein. * p<0.05 as compared to C57 mice evaluated by unpaired t-test.

FIGS. 10A-10D show PRMT4 inhibitor (TP-064) or neuron-specific AAV knockdown of PRMT4 reduced brain PRMT4 protein level in aged 3xTg-AD female mice. FIG. 10A shows TP-064 decreased PRMT4 in a dose-dependent manner. FIG. 10B is a fluorescent image of the coronal section of the brain 30 days after a single retro-orbital injection of GFP labelled AAV-PHP.eB expressing PRMT4-shRNA (PRMT4-AAV). FIGS. 10C and 10D show PRMT4-AAV reduced PRMT4 protein levels. * p<0.05 v. vehicle and #p<0.05 v. aged 3xTg-AD female mice were evaluated by one-way ANOVA with Tukey's post-hoc analysis (FIG. 10A) or unpaired t-test (FIGS. 10C and 10D).

FIGS. 11A-11D show neurovascular coupling dysfunction was alleviated with specific PRMT4 inhibitor (TP-064) or PRMT4-AAV knockdown in aged 3xTg-AD female mice. In FIG. 11A, neurovascular coupling was evaluated based on vascular tonicity changes in response to whisker stimulation (30 sec, 4 Hz) via two-photon laser scanning microscopy. FITC-dextran was injected IV for visualization of cerebral microvessels (Z=100 μm depth) in the cortex. In FIG. 11B, vessel diameters were measured before/after whisker stimulation, and before/after TP-064 (30 mg/kg for 7 days, IP) or PRMT4-AAV knockdown. In FIG. 11C, aged 3xTg-AD female mice had reduced microvessel diameter v. their male counterparts after whisker stimulation to suggest neurovascular coupling dysfunction in females. In FIG. 11D, microvessel diameters were increased in the presence of TP-064 or PRMT4-AAV knockdown to suggest recovery of neurovascular coupling. C57 mice treated with NOTCH1 inhibitor (Crenigacestat, 10 mg/kg, 7 days, IP) showed decrease in microvessel diameter to suggest neurovascular coupling deficiency similar to aged 3xTg-AD female mice. *p<0.05 compared to aged 3xTg-AD males (C) or C57 (D), #p<0.05 compared to 3xTg-AD+TP-064, $p<0.05 compared to 3xTg-AD+AAV, by unpaired t-test (C) or one-way ANOVA with Tukey's post-hoc analyses (D).

FIGS. 12A-12B show PRMT4 inhibitor (TP-064) enhanced regional cerebral blood flow in aged 3xTg-AD female mice. FIG. 12A is representative flux images of cortical vasculature via laser speckle contrast imaging. FIG. 12B shows aged 3xTg-AD female mice had impaired cortical cerebral blood flow as compared to age/sex-matched control C57 mice, while treatment with TP-064 (30 mg/kg, 7 days, IP) enhanced cerebral blood flow. * p<0.05 v. C57, #p<0.05 v. 3xTg-AD and C57+TP-064 evaluated by one-way ANOVA with Tukey's post-hoc analysis.

FIGS. 13A-13D are capillary-based immunoassay and associated graphs showing NOTCH1 protein expression was significantly decreased in the brain of aged 3xTg-AD female mice, while treatment with specific PRMT4 inhibitor (TP-064) or AAV knockdown of brain PRMT4 enhanced NOTCH1 protein level. NOTCH1 protein level in cortex, FIG. 13A, and hippocampus, FIG. 13B, was decreased in aged 3xTg-AD female mice v. male counterparts. Treatment with specific PRMT4 specific inhibitor (TP-064, 30 mg/kg, 7 days, IP) or AAV knockdown of PRMT4 enhanced NOTCH1 protein level in cortex, FIG. 13D, and hippocampus, FIG. 13D, in aged 3xTg-AD female mice. Relative NOTCH1 protein (˜110 kDa) level was measured by capillary-based immunoassay and normalized to total protein. * p<0.05 v. control male mice evaluated by unpaired t-test in FIGS. 13A and 13B. * p<0.05 compared to C57, #p<0.05 compared to 3xTg-AD+TP-064, $p<0.05 compared to 3xTg-AD+AAV, evaluated by one-way ANOVA with Tukey's post-hoc analyses in FIGS. 13C and 13D.

FIGS. 14A-14C show BBB leakage is reduced with specific PRMT4 inhibitor (TP-064) or AAV knockdown of PRMT4 in aged 3xTg-AD female mice. Aged female 3xTg-AD mice had higher levels of 40 kDa FITC-dextran in the perivascular space v. C57 controls. This was reduced with administration of PRMT inhibitor (TP-064) or AAV knockdown of PRMT4. FIG. 14A shows representative images of FITC to visualize brain microvessels and leakage into the perivascular space. FIG. 14B is a schematic representation of one of the disclosed experimental paradigms. FIG. 14C shows a graph of calculations of perivascular fluorescence based on the average of 3 ROIs per mice before and after 30 min of 40 kDa FITC-dextran administration. * p<0.05 v. C57, #p<0.05 v. 3xTg-AD female, evaluated by one-way ANOVA with Tukey's post-hoc analysis.

FIG. 15 is a graph showing blood brain barrier permeability was impaired in aged 3xTg-AD female mice but reversed with TP-064. Aged 3xTg-AD female mice+TP-064 had lower brain penetration of Evan's blue (decreased BBB leakage) v. aged 3xTg-AD female mice. Formamide extraction of Evan's blue infused-whole-brain was quantified. * p<0.05 compared to C57, #p<0.05 compared to 3xTg-AD, evaluated by one-way ANOVA with Tukey's post-hoc analysis.

FIGS. 16A-16G show NOTCH1 inhibition (via crenigacestat) decreased ZO-1 and occluding and AAV knockdown of PRMT4 enhanced ZO-1 and occludin. FIG. 16A shows RNAseq heatmap data evidencing that decreased junctional proteins in aged 3xTg-AD v. C57 female mice. FIGS. 16B and 16C show crenigacestat (10 mg/kg, IP, 7 days) reduced NOTCH1 (110 kDa) protein level in the cortex and hippocampus of aged C57 female mice. AAV knockdown of PRMT4 increased NOTCH1 protein level. FIGS. 16D-16G show ZO-1 (220 kDa), and occludin (60 kDa) tight junctional proteins were decreased in the cortex and hippocampus in presence of NOTCH1 inhibitor in C57. PRMT4-AAV increased the expression of ZO-1 and occludin in aged 3xTg-AD female mice. Protein level was measured by capillary-based immunoassay and normalized to total protein. * p<0.05 compared to C57, #p<0.05 compared to 3xTg-AD, $p<0.05 compared to 3xTg-AD+AAV as evaluated by one-way ANOVA with Tukey's post-hoc analyses.

FIGS. 17A-17D show leukocyte rolling velocity was reduced in aged 3xTg-AD female mice. FIG. 17A shows two-photon laser scanning microscopy images of rolling leukocytes (labeled with acridine orange, red circle) in cortical microvessels.

FIG. 17B shows aged 3xTg-AD female mice have decreased leukocyte velocities v. aged 3xTg-AD male and C57 female mice, results shown graphically in FIGS. 17C and 17C. Leukocytes velocity measurements were performed by linescans (512 lines/sec). *p<0.05 compared to aged 3xTg-AD male or C57 female evaluated by unpaired t-test. FIGS. 18A and 18B are capillary-based immunoassay and associated graphs showing PRMT4 inhibitor (TP-064) or AAV knockdown of PRMT4 reduced GFAP levels in aged 3xTg-AD female mice. Glial fibrillary acidic protein (GFAP, 55 kDa) was increased in aged 3xTg-AD female mice v. C57, which indicates neuroinflammation, but reversed with TP-064 (30 mg/kg, 7 days, IP) or PRMT4-AAV treatment in cortex, shown in FIG. 18A, and hippocampus, shown in FIG. 18B. Protein level was measured by capillary-based immunoassay and normalized to total protein. *p<0.05 as compared to C57, #p<0.05 v. 3xTg-AD evaluated by one-way ANOVA with Tukey's post-hoc analyses.

FIGS. 19A-19F are six graphs showing PRMT4 inhibitor (TP-064) reduced ICAM-1, VCAM-1 and enhanced E-Cadherin protein levels in aged female 3xTg-AD mice. In FIGS. 19A-19D, aged 3xTg-AD female mice v. C57 had increased ICAM-1 and VCAM-1 levels, which were decreased with TP-064 (30 mg/kg, 7 days, IP) suggesting more leukocyte adhesion. In FIGS. 19DE and 19F, aged 3xTg-AD female mice had lower E-Cadherin level, that was elevated with TP-064 (30 mg/kg, 7 days, IP) treatment to suggest more cell-cell adhesion. Adhesion molecules were assessed via protein chip assay in the cortex and hippocampus. * p<0.05 evaluated by one-way ANOVA with Tukey's post-hoc analyses.

FIGS. 20A and 20B are two graphs showing PRMT4 inhibitor (TP-064) or AAV knockdown of PRMT4 improved functional learning/memory in aged 3xTg-AD female mice. FIG. 20A shows T-maze spontaneous alternation ratio decreased in aged 3xTg-AD female mice v. C57 but increased with administration of TP-064 (30 mg/kg, 7 days, IP). In FIG. 20B, in Novel Object Recognition, aged 3xTg-AD female mice had fewer number of entries in the novel zone v. C57. Treatment with TP-064 or brain-specific AAV knockdown of PRMT4 increased the number of entries. T-maze and novel object recognition test were utilized to analyze working/short-term and reference/long-term memory, respectively. * p<0.05 v. C57 via one-way ANOVA with Tukey's post-hoc analyses.

FIGS. 21A and 21B shows contextual learning impairment was improved in AAV knockdown of PRMT4 in aged 3xTg-AD female mice. In FIG. 21A, the two-photon laser scanning microscopy imaging of neurovascular coupling was assessed in the whisker-barrel cortex intended to detect deficits in contextual learning that are whisker dependent. Whisker stimulation-dependent perceptual learning was assessed using a novel texture discrimination task known to be dependent on somatosensory cortex activity. Wooden blocks of the same dimensions but wrapped in different sandpaper (60 v. 150 grit) were placed in the box. FIG. 21B shows with aged 3xTg-AD female mice, time spent in novel texture zone was significantly decreased as compared to aged C57 female mice and improved by PRMT4-AAV knockdown. * p<0.05 v. C57 and $p<0.05 vs. 3xTg-AD+AAV via one-way ANOVA with Tukey's post-hoc analyses.

FIG. 22 is a chemical structure of TP-064.

FIGS. 23A-23E show aged female 3xTg-AD mice exhibit differential expression of PRMT4 which can be reversed by TP-064. Relative mRNA levels and protein of PRMTs in the cortex and hippocampus were measured by quantitative RT-PCR and capillary-based immunoassay, respectively. FIG. 23A shows Relative mRNA levels of PRMTs 1-9 were measured in brain tissue from aged, female C57 and 3xTg mice. FIGS. 23B and 23C show relative protein expression of protein arginine methyltransferase 6 (PRMT6) (˜45 kDA) (FIG. 23B) and PRMT4 (˜63 kDA) (FIG. 23C) were measured via ProteinSimple capillary-based immunoassay in cortical and hippocampal lysates from aged, female C57 and 3xTg animals. FIG. 23D shows computer generated pseudo-blot images depicting representative bands for PRMT4 and PRMT6. FIG. 23E shows PRMT4 inhibitor TP-064 reduced PRMT4 protein expression in a dose-dependent manner. Results are expressed as mean+/−SEM, n indicates number of animals used. * p<0.05, ** p<0.02, *** p<0.01, v. control or untreated 3xTg, evaluated by one-way ANOVA with Bonferroni post-hoc analysis and Student's t-test as appropriate, (n=3-6).

FIGS. 24A-24D show aged female 3xTg mice have higher levels of brain amyloid precursor protein and phospho-tau compared to age- and sex-matched C57 mice. Relative protein levels of APP in the cortex (FIG. 24A) and hippocampus (FIG. 24B) were measured by capillary-based immunoassay. Relative protein levels of pTau in the cortex (FIG. 24C) and hippocampus (FIG. 24D) were measured by capillary-based immunoassay. Psuedo-blot images were generated using peak values in Compass for SW software. Results were normalized with total protein *p<0.05 v. C57 as evaluated by one-way ANOVA with Tukey's post-hoc analysis.

FIGS. 25A-25D show aged female 3xTg-AD mice have increased levels of asymmetric dimethylarginine (ADMA), enhanced expression of dimethylarginine dimethylaminohydrolase 2 (DDAH2), and increased levels of peroxynitrite derivative 3-nitrotyrosine. FIG. 25A shows ADMA was measured via ELISA. Results suggest elevated ADMA in mouse 3xTg EDTA-plasma compared to age/sex-matched controls. FIG. 25B shows quantified relative expression of DDAH2 protein in aged C57 and 3xTg mice with pseudo-blot images of DDAH2 bands obtained from capillary-phoresis (ProteinSimple). FIG. 25C shows a schematic illustration of superoxide generating ONOO·⁻ and nitric oxide. In turn, ONOO·⁻ causes tyrosine nitration, forming 3-nitrotyrosine. FIG. 25D shows 3-nitrotyrosine concentration was obtained via ELISA. Our results suggest that 3-nitrotyrosine was elevated in 3xTg combined cortex and hippocampus lysate to suggest enhanced level of peroxynitrite generation. *** p<0.05 v. control and 3xTg, *p<0.05 v. 3xTg as evaluated by one-way ANOVA with Tukey's post-hoc analysis, results are expressed as mean+/−SEM, n indicates number of animals used, (n=3-7).

FIGS. 26A-26E show nitric oxide synthase function is impaired in aged female 3xTg-AD mice. FIG. 26A shows NO metabolites nitrite (NO₂) and nitrate (NO₃) were measured using a Griess Reaction ELISA kit (Cat. No. 780001, Cayman Chemical, Ann Arbor, MI) from whole brain tissue lysate. FIG. 26B shows computer generated pseudo-blot images depicting representative bands for eNOS, nNOS, and iNOS obtained from capillary-phoresis (ProteinSimple). FIG. 26C shows quantified relative expression of eNOS protein in aged C57 and 3xTg mice. FIG. 26D shows quantified relative expression of nNOS protein in aged C57 and 3xTg mice. FIG. 26E shows quantified relative expression of iNOS protein in aged C57 and 3xTg mice. Results are expressed as mean+/−SEM, *p<0.05 v. C57 evaluated by one-way ANOVA with Tukey's post-hoc analysis, n indicates number of animals used, data was calculated from the ratio between the areas of the protein target of interest and total protein area X 1000 (n=3-9).

FIGS. 27A and 27B show PRMT4 inhibition via TP-064 enhances regional cortical cerebral blood flow. Representative flux image of cortical vasculature via laser speckle contrast imaging (FIG. 27A). Aged 3xTg-AD female mice had impaired cortical regional cerebral blood flow as compared to age/sex-matched control C57 mice, while treatment with TP-064 (30 mg/kg, 7 days, IP) enhanced cerebral blood flow (FIG. 27B). Results are expressed as mean+/−SEM, n indicates number of animals used, *p<0.05 v. C57, ^{#}p 0.05 v. 3xTg and C57±TP-064 evaluated by two-way ANOVA with Tukey's post-hoc analyses.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means that the property is within 80% of its desired value. In other embodiments, “substantially” means that the property is within 90% of its desired value. In other embodiments, “substantially” means that the property is within 95% of its desired value. In other embodiments, “substantially” means that the property is within 99% of its desired value. For example, the term “substantially complete” means that a process is at least 80% complete, for example. In other embodiments, the term “substantially complete” means that a process is at least 90% complete, for example. In other embodiments, the term “substantially complete” means that a process is at least 95% complete, for example. In other embodiments, the term “substantially complete” means that a process is at least 99% complete, for example.

The term “substantially” includes a value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

The term “about” includes when value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

In addition, the invention does not require that all the advantageous features and all the advantages of any of the embodiments need to be incorporated into every embodiment of the invention.

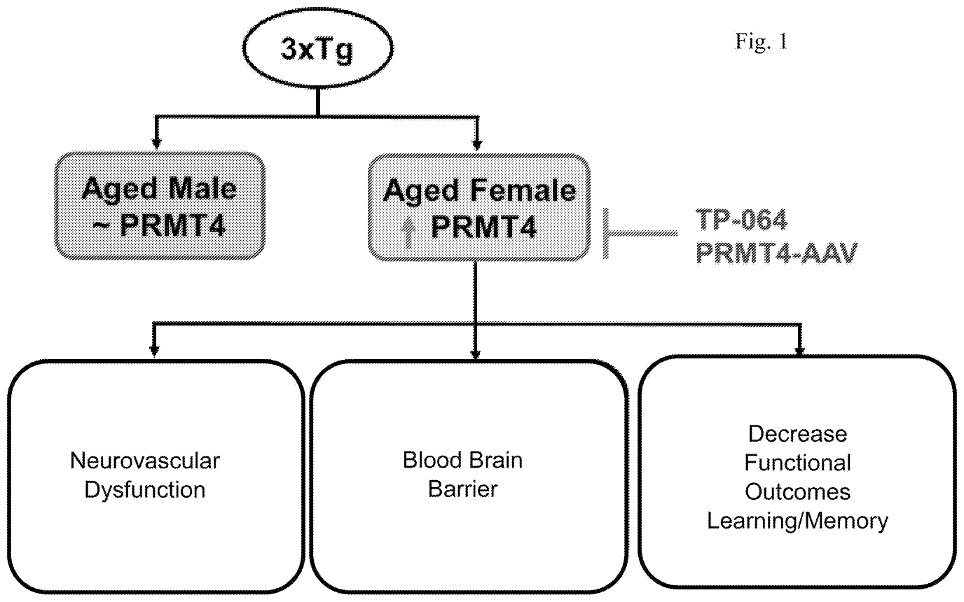

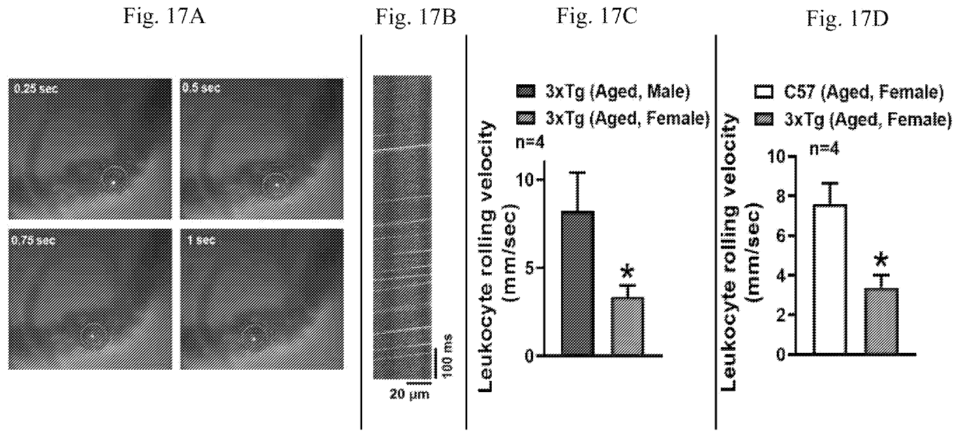

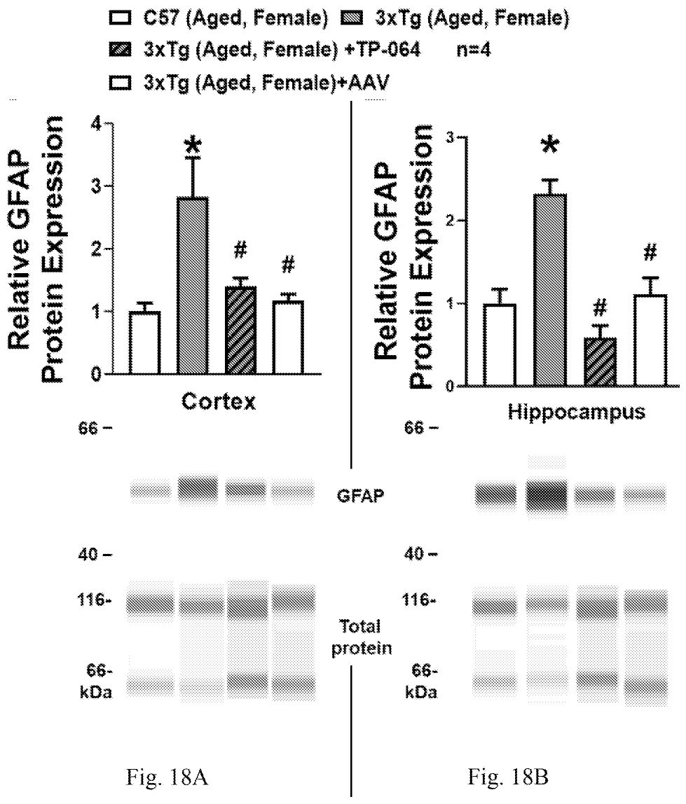

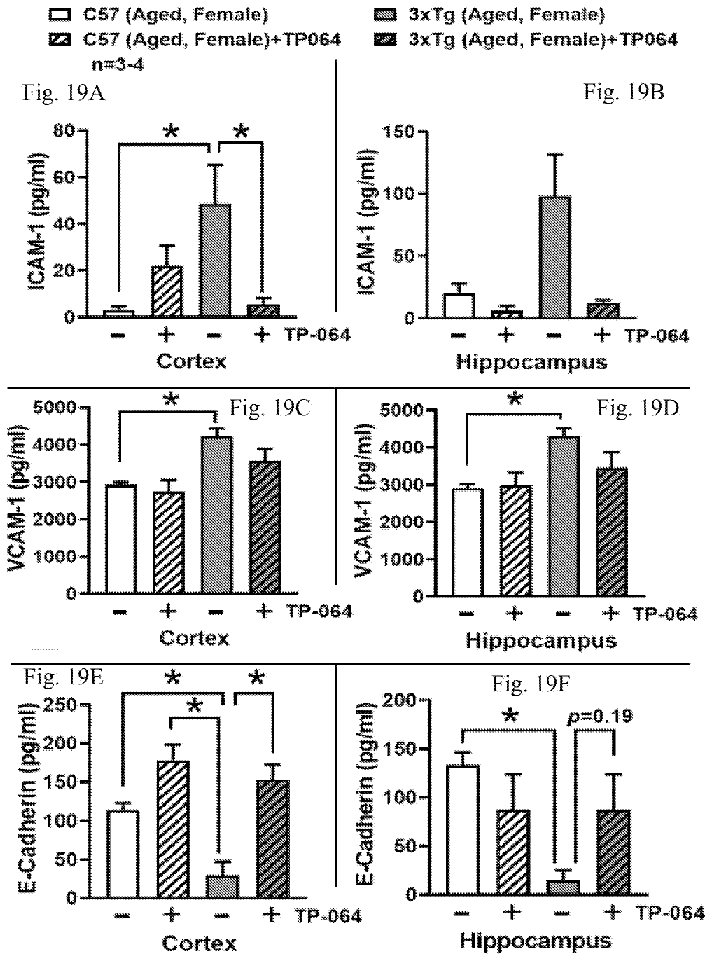

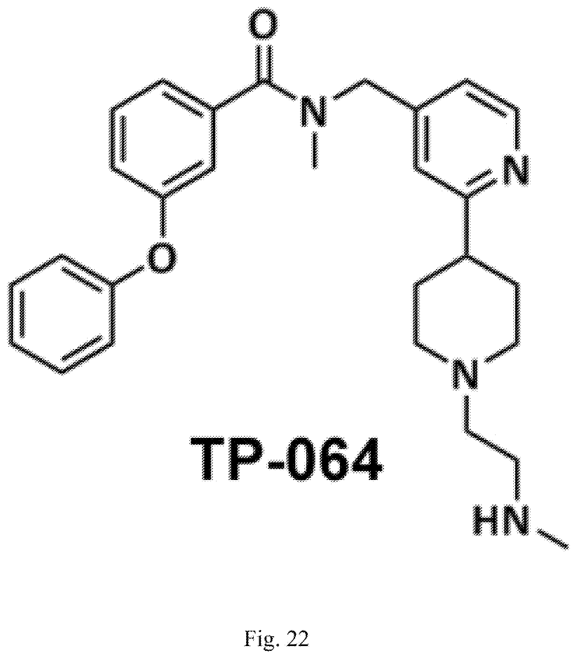

Turning now to FIGS. 1-22, a brief description concerning the various components of the present invention will now be briefly discussed.

While AD is associated with tau and β-amyloid accumulation, concurrent derangements in cerebral blood flow have been observed alongside these proteinopathies. Since AD involves neurofibrillary tangles due to tau aggregation and β-amyloid accumulation resulting in deteriorated cognition, the inventors used the aged 3xTg-AD male/female mouse to recapitulate the development of AD in humans.

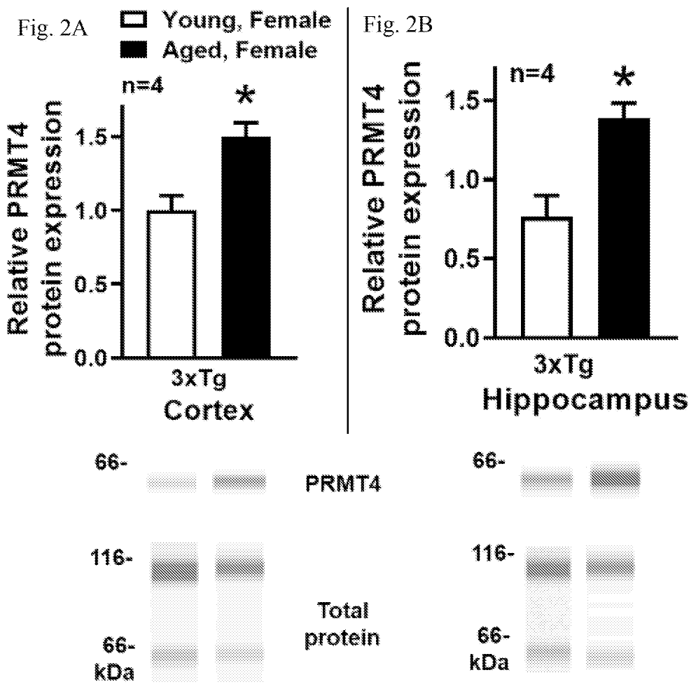

Targeting novel pathways which contribute to AD progression is crucial due to a lack of treatment modalities, and novel targets can offer alternative therapies for the treatment of AD. The inventors are aware that protein arginine methyltransferase (PRMT) 4 (also known as coactivator-associated arginine methyltransferase 1; CARM1), part of a novel family of enzymes (PRMT1-9), has been implicated in cancer and immunity/inflammation, but there has been limited investigation and knowledge of its neurovascular function. Decreased PRMT4 levels are associated with loss of astroglial development and is embryonically lethal in knockout mice. Since PRMT4 is enhanced in aged 3xTg-AD female mice v. their younger and male counterparts, it makes the age-dependent expression of PRMT4 enzyme a novel and relevant target against AD, as shown in FIGS. 2A-3C, and 5A-5D.

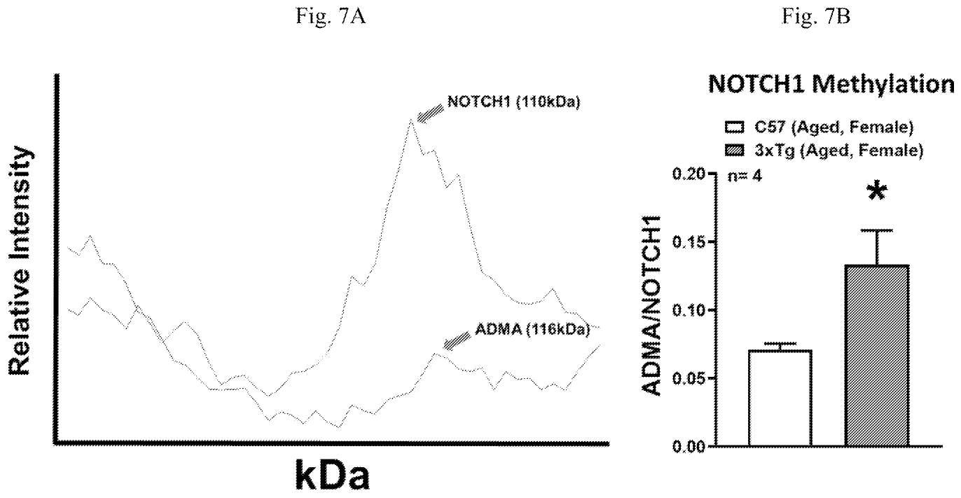

Aspects of the disclosed experimentation: A) Characterization of the age and sex-dependent role of PRMT4 in AD. B) Identification of PRMT4 as a regulator of NOTCH1 signaling. The inventors' data evidences that PRMT4 can methylate the intracellular domain of NOTCH1 leading to its degradation, and results in poor functional outcomes in aged 3xTg-AD female mice as shown in FIGS. 7A, 7B, and 20A-21B. NOTCH1 is lower in patients with AD and can diminish blood brain barrier (BBB) integrity. C) Showing that TP-064 (specific PRMT4 inhibitor) or AAV to decreased PRMT4 (PRMT4-AAV) to target PRMT4 in AD.

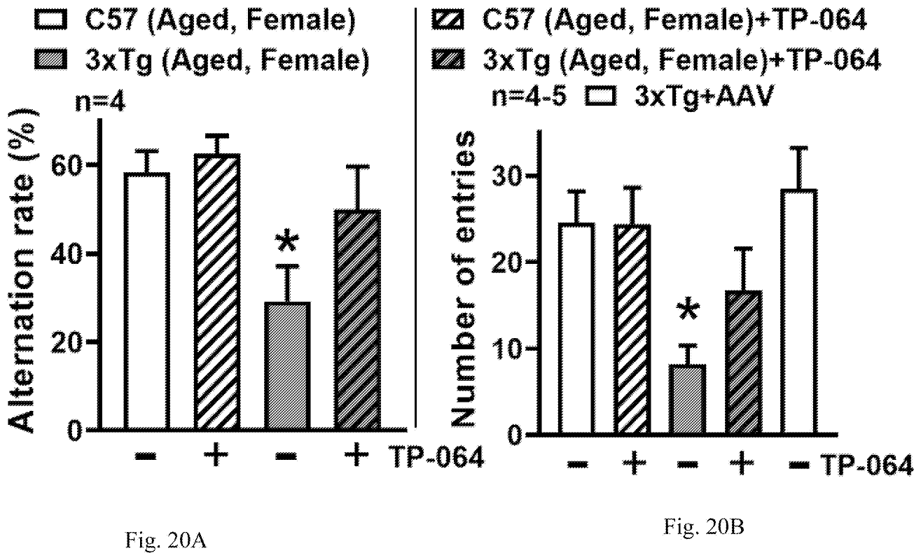

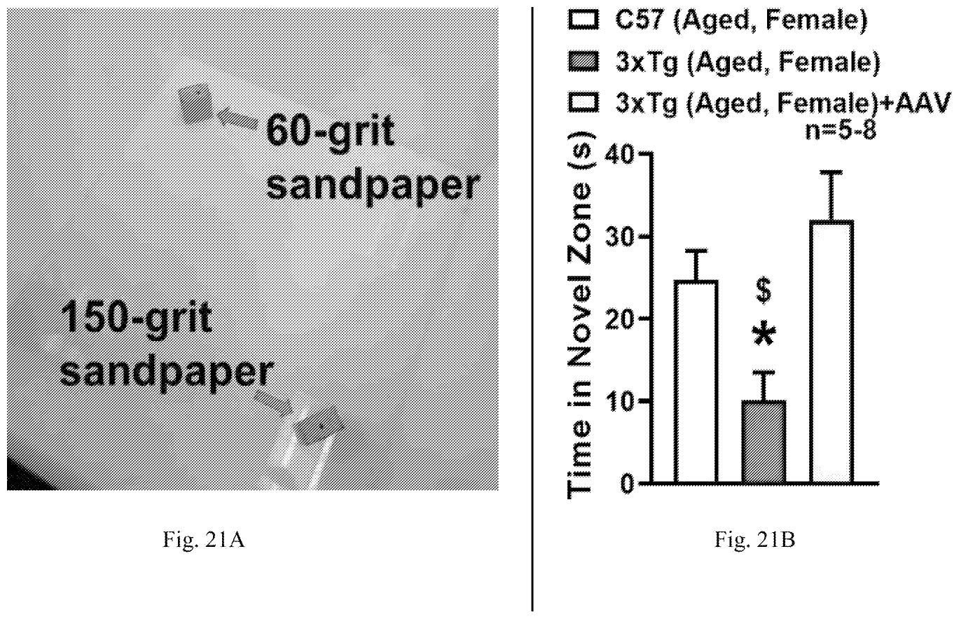

The inventors' disclose PRMT4-NOTCH1 as an important modulator of age and sex-dependent progression of AD pathology. Utilizing sex and aged matched 3xTg-AD mice, the inventors' data evidences that PRMT4 is enhanced in aged 3xTg-AD female mice v. their young and male counterparts, as shown in FIGS. 2A-3C, and 5A-5D. This overall enhancement of PRMT4 levels disrupts 1) neurovascular coupling, 2) blood brain barrier (BBB) and 3) functional learning and memory, as shown in FIGS. 20A-21B. The inventors conclude based on the data that inhibition of PRMT4 (TP-064 or PRMT4-AAV, for example) enhances neurovascular coupling, maintain BBB integrity, and preserving functional learning/memory in aged 3xTg-AD female mice.

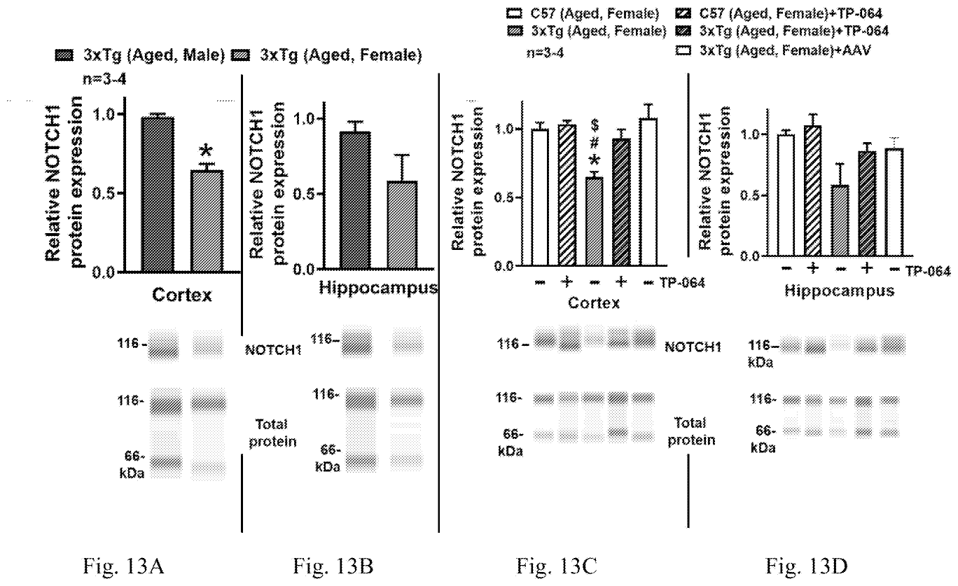

Inquiry±1: Determining if PRMT4 can modulate neurovascular coupling. The inventors posited the hypothesis that aged 3xTg-AD female mice have enhanced PRMT4 protein levels resulting in decreased neurovascular coupling. One rationale was that aged 3xTg-AD female mice have enhanced PRMT4 mRNA and protein levels v. aged 3xTg-AD male and non-AD C57BL/J6 (C57) counterparts. The inventors inhibited PRMT4 (TP-064, specific PRMT4 inhibitor, or PRMT4-AAV) to investigate neurovascular coupling by whisker-barrel stimulation coupled with two-photon laser scanning microscopy and laser speckle contrast imaging to measure cerebral blood flow and vessel diameter. The inventors showed that inhibition of PRMT4 (via TP-064 or PRMT4-AAV) 1) increased cerebral blood flow and vessel diameters, and 2) increased neurovascular coupling. Since PRMT4 methylates NOTCH1, as shown in FIGS. 7A, 7B, and 13A-13D, the inventors also determined NOTCH1-mediated neurovascular coupling as a mechanism of action.

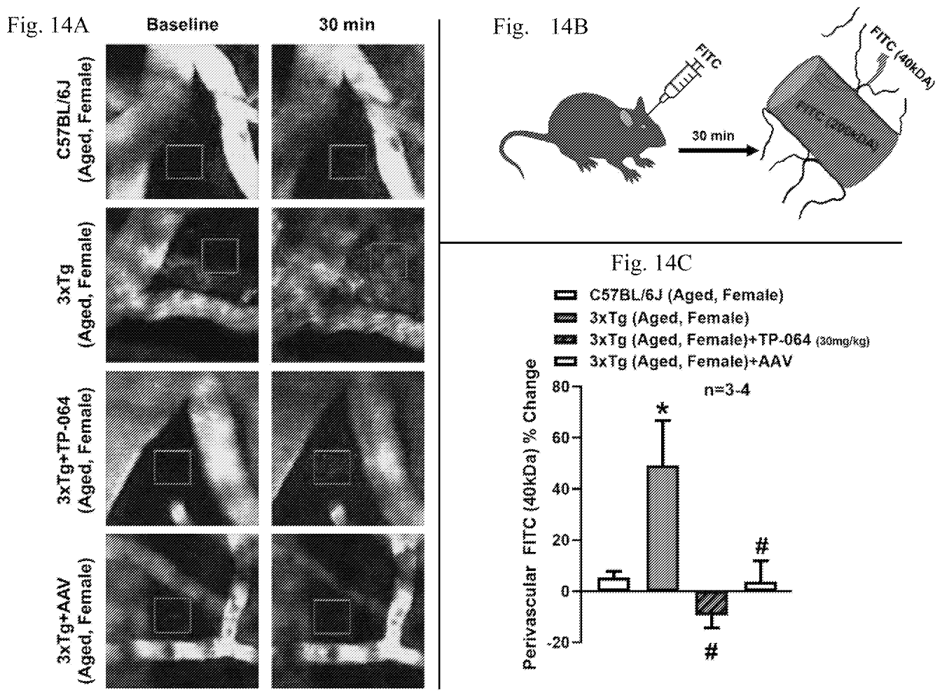

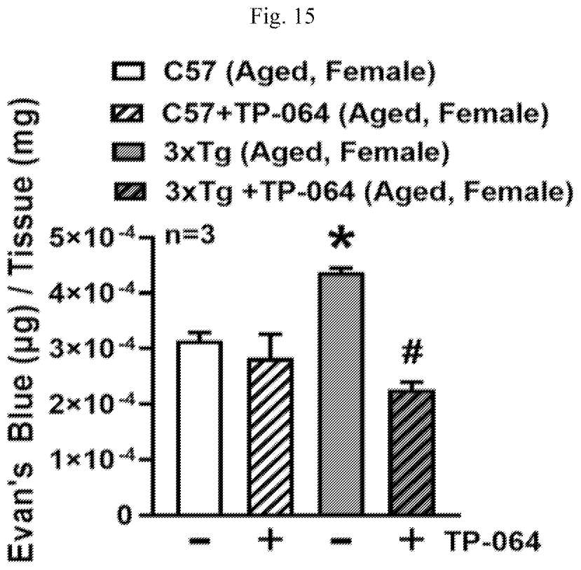

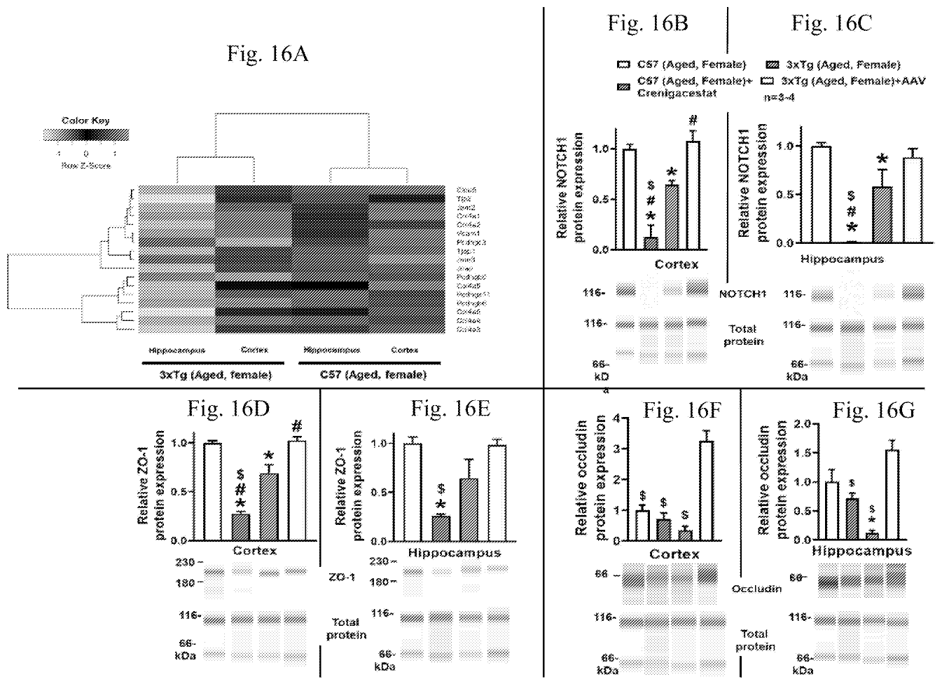

Inquiry±2: Determining if PRMT4 can modulate the blood brain barrier (BBB). The inventors posited the hypothesis that enhanced PRMT4 can increase BBB permeability (leakage), while decreased PRMT4 via TP-064 or PRMT4-AAV can diminish BBB permeability in aged 3x-Tg-AD female mice. One rationale was that cerebral vascular dysfunction can exacerbate BBB disruption in the aged AD brain. Aged 3xTg-AD female mice have impaired BBB permeability (more leakage), which is reduced with TP-064 or PRMT4-AAV, as shown in FIGS. 14A-15. These female mice also have lower expression of tight junction proteins (ZO-1, occludin), to evidencing that BBB is compromised, as shown in FIGS. 16A-16G, which modulates leukocyte infiltration. The inventors showed that inhibition of PRMT4 (TP-064 or PRMT4-AAV) 1) decreased BBB leakage, 2) increased leukocyte rolling velocity, and 3) restored adhesion molecule profiles.

Inquiry±3: Determining if PRMT4 can modulate learning/memory impairments. The inventors hypothesized that enhanced PRMT4 levels of aged 3xTg-AD female mice can decrease functional learning/memory. One rationale was that inhibition of PRMT4 can enhance contextual learning and memory (via T-maze, novel object recognition, and novel texture discrimination task), as shown in FIGS. 20A-21B. The inventors showed that aged 3xTg-AD female mice have overall decreased functional learning/memory, which is reversed with TP-064 or PRMT4-AAV knockdown.

Alzheimer's disease (AD) is characterized by neurofibrillary tangles due to tau aggregation and plaques mediated by β-amyloid accumulation resulting in deteriorated cognition. Targeting novel pathways or physiological mechanisms which contribute to AD progression is crucial due to a lack of treatment modalities in the current technology. Older women are more likely to develop a rapid progression of AD than men when predictive factors such as obesity, lifespan, and elevated stroke severity are considered. While AD is associated with tau and β-amyloid accumulation, concurrent derangements in cerebral blood flow (CBF) have been observed alongside these proteinopathies in humans and mice. The inventors used the aged 3xTg-AD male and female mouse model due to the overexpression of both β-amyloid and tau to recapitulate features of human AD pathology. Attenuation of neurovascular coupling is associated with the progression of AD. The mismatch in cerebral perfusion and metabolic demand contributes to the neurodegeneration and atrophy that is a hallmark of AD. Additionally, in the aged AD brain, cerebral vascular dysfunction can impair blood brain barrier (BBB) integrity, as well as cause loss of junctional proteins. BBB dysfunction creates a permissive environment for leukocyte infiltration, initiating the chronic neuroinflammation associated with AD, as well as neurodegeneration/cognitive decline.

Targeting novel pathways which contribute to AD progression is crucial due to a lack of treatment modalities, and novel targets can offer alternative therapies for the treatment of AD. One such target for the inventors was in the protein arginine methyltransferase 4 (PRMT4), part of a novel enzyme family that plays roles in cancer and immunity/inflammation, but with limited knowledge of neurovascular function. PRMTs are responsible for the methylation of arginine residues, which is a post-translational modification involved in mRNA splicing, DNA repair, signal transduction, protein interaction, and transport. However, much remains to be elucidated in their basic neurological function. This novel and difficult area of inquiry has only been marginally addressed, with limited implications in neurological diseases There are 9 PRMT isoforms (PRMT1-9) and the inventors' focus was on protein arginine methyltransferase 4 (PRMT4). PRMT4 is important in neurological development as its loss is associated with deranged astroglial lineage and is embryologically lethal in PRMT4 knockout mice. Since PRMT4 is enhanced in aged 3xTg-AD mice v. younger counterparts, this makes the age-dependent PRMT4 a novel and relevant target against AD, as shown in FIGS. 2A and 2B.

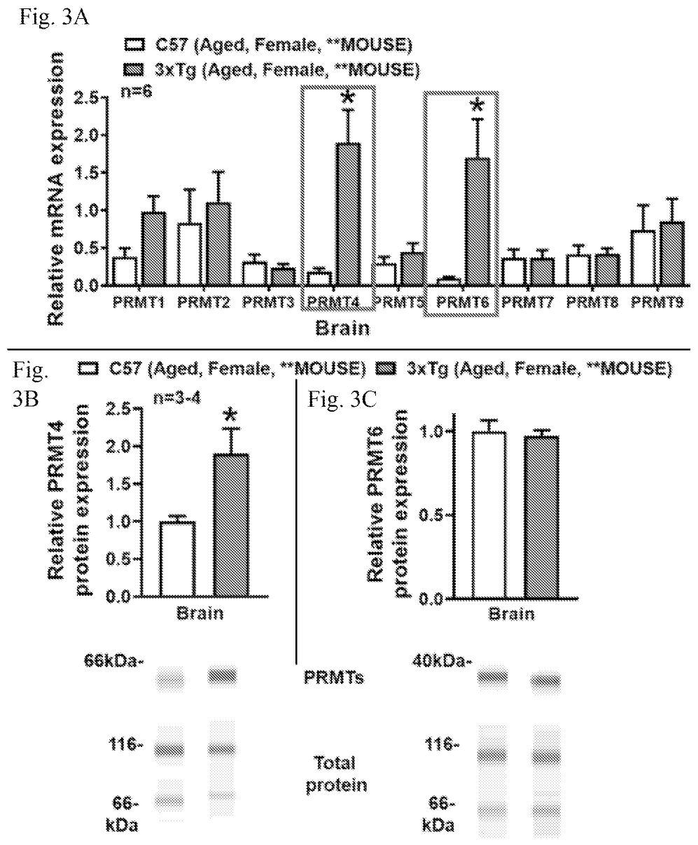

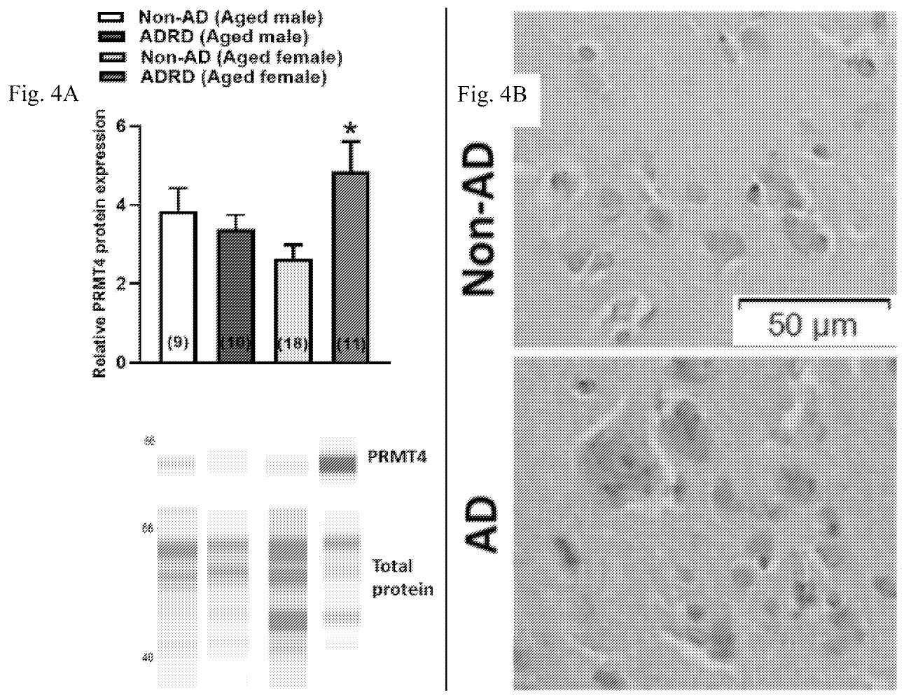

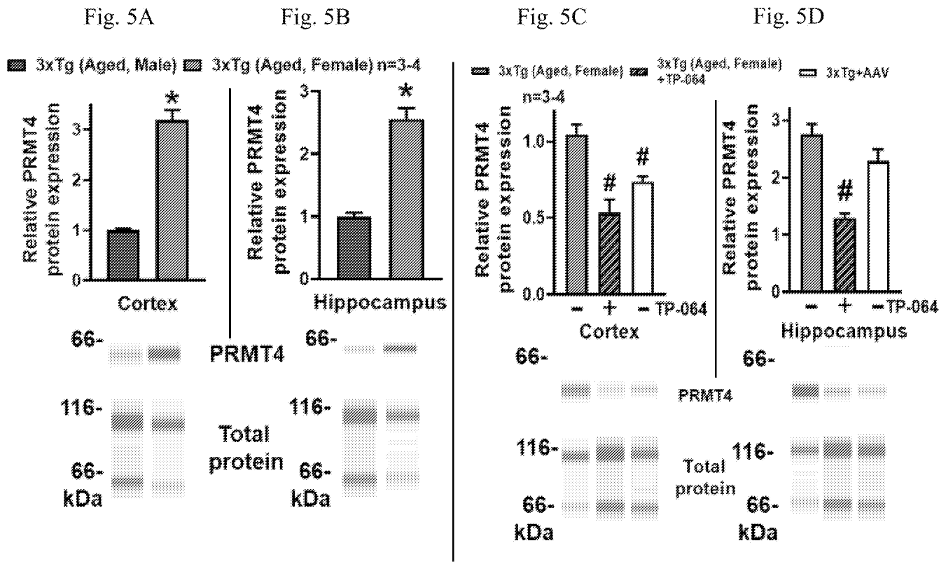

The inventors elected to focus on PRMT4 based on experimental results. The inventors initially surveyed PRMTs via RT-qPCR in the mouse brain with PRMT4 and PRMT6 mRNA being found elevated in 3xTg-AD females, as shown in FIG. 3A. However, PRMT4 but not PRMT6 protein was enhanced in 3xTg-AD females, as shown in FIGS. 3B and 3C, but not in males, as shown in FIG. 5A. This led the inventors to focus on age and sex differences with focus on PRMT4. Age-related increase of PRMT4 is brain specific, as such increase in PRMT4 was not observed in lungs of aged-3xTg female mice (data not shown). This is further corroborated by a separate experiment finding that PRMT4 protein increased ˜40% as compared to other PRMTs in whole-blood and brain tissue are enhanced in human AD females over 60 years old, as shown in FIGS. 4A and 4B. These studies evidenced strong enough that PRMT4 is highest in AD females to warrant further investigation.

From PRMT4 to NOTCH. A functional significance of PRMT4 in the CNS is currently unknown in the literature. PRMT4 is a specific methylator of the Proline, Glycine, Methionine motif present in the intracellular domain of NOTCH1. Methylation of this domain leads to its degradation. To further understand the biology of PRMT4 in AD the inventors posed the question, “What does PRMT4 methylate in the brain?” NOTCH family of receptors (NOTCH1-4) have been broadly implicated in various disease states including cerebral autosomal dominant arteriopathy with subcortical infarcts and leukoencephalopathy (CADASIL), a genetic small-vessel disease. NOTCH signaling has been implicated in AD including vascular angiogenesis activity, development, proliferation, differentiation, apoptosis, regeneration, and inflammation. Altered NOTCH1 expression has been associated with AD, as soluble level of NOTCH1 is lower in human AD patients. Knockdown of NOTCH1 can increase blood brain barrier permeability (more leakage) resulting in further brain damage. Thus, the intersection of the aforementioned neuronal and vascular NOTCH1 functions, combined with its propensity for site-specific methylation and regulation by PRMT4 present a novel and critical axis of AD pathology. The inventors' thus investigated PRMT4-NOTCH1 as an important modulator of age and sex-dependent progression of AD pathology.

Sex Specific. Cortical and hippocampal PRMT4 protein levels were enhanced in aged 3xTg-AD female, not male mice, but decreased with PRMT4 specific inhibitor (TP-064 and PRMT4-AAV), as shown in FIGS. 5A-5D. This is further corroborated with the inventors' RT-qPCR data to evidence that PRMT4 mRNA levels were enhanced in aged 3xTg-AD female mice, as compared to age and sex-matched C57BL/6J, as shown in FIGS. 3A-3C.

Species. 3xTg-AD mice have been shown to have a progressive increase in β-amyloid, phosphorylated tau, hippocampal and cortical plaques, and cognitive decline detected as early as 6 months of age. The inventors used aged 3xTg-AD male and female mouse models due to the overexpression of β-amyloid and tau. A single mouse model with both mutations of amyloid and tau (3xTg-AD) more accurately represents AD pathology v. single proteinopathy. 3xTg-AD are homozygous for 3 mutant alleles (Psen1, APPSwe, tauP301L) producing a clinically relevant model for AD, with consistent and reproducible data within a reasonable timeline (˜9-12 months), v. longer time-points in other AD models of porcine (>1 year), and primates (>10 years). The model is not perfect though, as neurodegeneration, observed in humans, has not been shown in 3xTg-AD mice.

Young v. Aged. To further show that this is an age-related phenomenon, aged 3xTg-AD female mice presented enhanced PRMT4 protein levels, as compared to young 3xTg-AD or C57BL/6J female mice, as shown in FIGS. 2A-3C. Altogether, the inventors' preliminary results suggest that PRMT4 is enhanced in aged 3xTg-AD female mice, but not in male mice, as shown in FIGS. 5A-5D. While the age range of 9-12 months is not canonically considered “aged”, this is a crucial period in the lifespan of the 3xTg-AD strain, characterized by hallmarks of accumulation of β-amyloid and tau protein, coupled with worsened cognitive deficits. This is an important, clinically relevant juncture of pre-AD to early AD, which places emphasis on preventing further progression of dementia, and not just treating the later stages of AD.

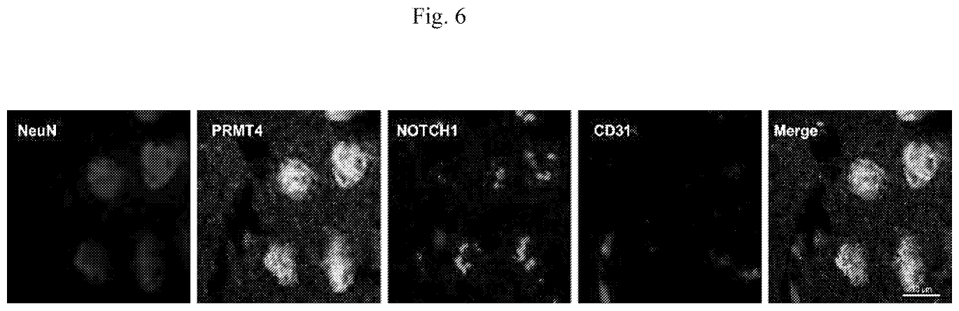

The inventors focused on NOTCH1 rather than the other NOTCH isoforms as NOTCH1 is the only member that is methylated by PRMT4 in its NOTCH Intracellular Domain (NICD) at five conserved arginine residues in the C-terminal, which are not present in NOTCH2-4. The inventors' data suggest that PRMT4 and NOTCH1 are co-expressed in perivascular neurons, as shown in FIG. 6. In fact, PRMT4 enhances NOTCH1 methylation, demonstrated by increased asymmetric dimethylarginine (ADMA) levels as shown in FIGS. 7A and 7B, leading to rapid degradation of NICD. PRMT4 methylates arginine residues to produce asymmetric dimethylarginine (ADMA). The inventors measured ADMA levels as an indicator of methylation.

In the experiments, the inventors characterized the role of PRMT4 in age and sex-dependent AD, identified PRMT4 as a regulator of NOTCH1 in neurovascular signaling and blood brain barrier permeability, and evaluated PRMT4 modulation via TP-064 (specific PRMT4 inhibitor) or PRMT4-AAV knockdown as a target for AD. The inventors also used two-photon laser scanning microscopy for the real-time measurement of cerebral blood flow/vessel diameters and leukocyte rolling. The inventors used well-established methodology to evaluate neurovascular paradigms: whisker-barrel stimulation in conjunction with two-photon laser scanning microscopy and laser speckle contrast imaging. BBB parameters (tight junction proteins), and functional outcomes measures, such as the novel texture discrimination task to corroborate the inventors' neurovascular coupling findings and demonstrated through rigorous preliminary data.

Sex as a Biological Variable: The incidence of AD increases with age, with aged women at even greater risk. Pathology-specific mechanisms to explain this sex difference in AD remain unknown. The cessation of the estrous cycle with aging (estropause) in mice is analogous to menopause in humans, and as AD is regarded as an age-related disease, it is thought that post-reproductive aging in females contributes to dementia. According to the American Associate of Neurology, “Women who had given birth to five or more children were 70% more likely to develop AD than women who gave birth to fewer children.” Based on this and other related studies, The inventors considered the usage of aged (9-12 month old) female 3xTg-AD, which have been designated as “retired breeders” by JAX to model more severe AD. This life-phase is equivalent to late middle-aged menopause in humans. Aged (9-12 month old) female C57BL/6J will be designated as control, repeated in age-matched male 3xTg-AD mice.

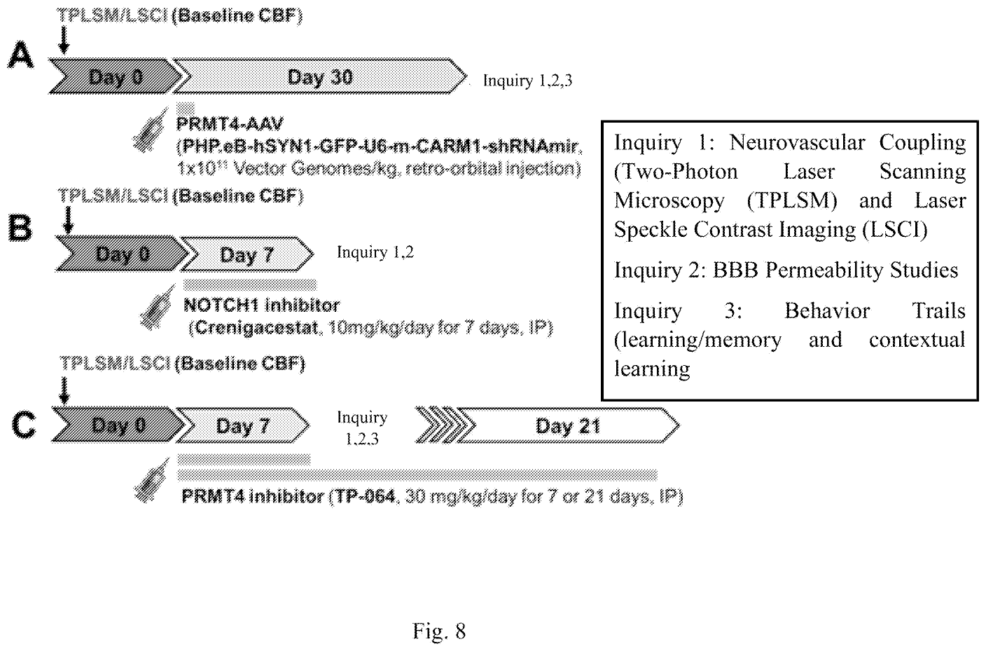

Scientific Rigor: The inventors' proposed study was performed in an unbiased and rigorous manner. All animals were purchased from JAX (3xTg-AD, Strain±004807, C57BL/6J). All drugs (TP-064 and Crenigacestat), antibodies were commercially available. Crenigacestat concentrations were derived from previous studies in the field as well as the inventors' dose response trials purposefully designed to attenuate NOTCH1 protein levels. All investigators were blinded to the studies, and all animals were randomized to different treatment groups. Detailed experimental protocol is presented in FIG. 8 and the groups outlined in the Vertebrate Animal Subjects.

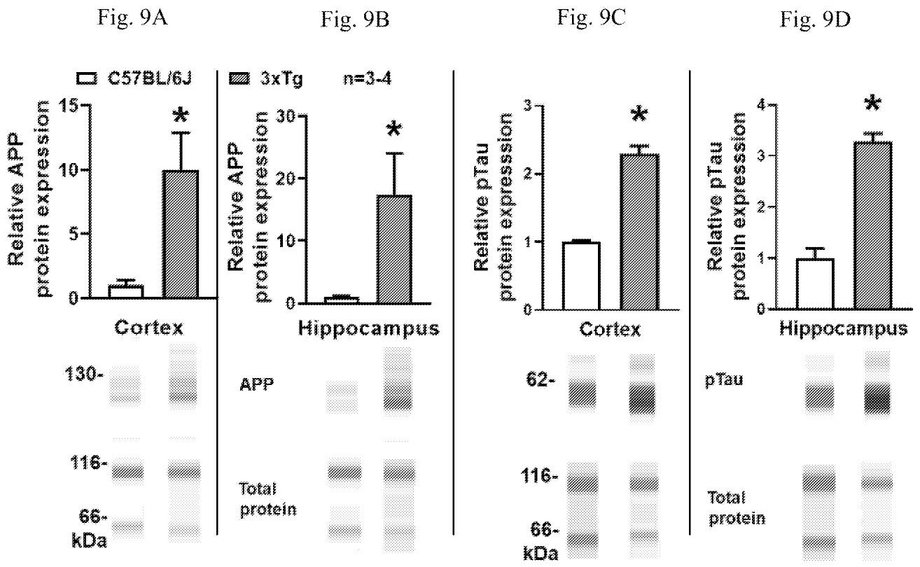

Experiments were conducted on a genetic background of 3xTg-AD female mouse (C57BL/6J were used as controls). The 3xTg-AD mice suggest a progressive increase in β-amyloid, phosphorylated tau, hippocampal and cortical plaques, and cognitive decline detected at 6 months of age. Aged (9-12 months old) female C57BL/6J are cognitively normal without deficits. The inventors measured APP and pTau as shown in FIGS. 9A-9D to assess overall AD pathology coupled with functional behavioral. The inventors administered specific PRMT4 inhibitor (TP-064, 30 mg/kg/day, IP) daily for 7(acute) or 21(chronic) days or PRMT4-AAV (single injection, 30-day incubation) in aged C57 and 3xTg-AD female mice.

Methods/biological/chemical resources used were well-established such as two-photon laser scanning microscopy, laser-speckle contrast imaging, protein arrays, real-time reverse transcription-PCR, Protein Simple Jess (capillary-based immunoassay) for the evaluation of proteins, and immunohistochemistry.

The experiments examined the hippocampus/cortex due to the fact that behavioral T-maze [learning/memory, (hippocampus)] is a functional endpoint, and that cerebral tonicity/blood flow and leukocyte rolling experiments take place in the cortex via two-photon laser scanning microscopy and laser speckle contrast imaging.

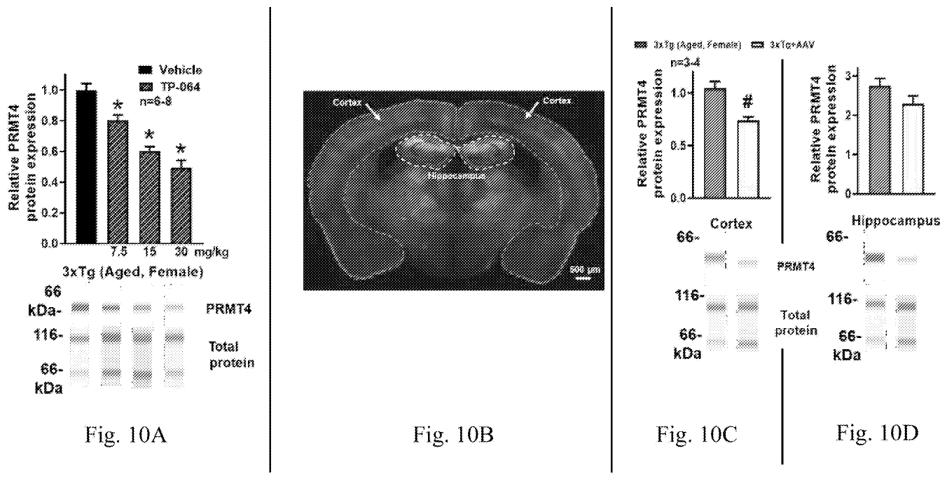

Pharmacological approach: TP-064 is a potent and selective PRMT4 inhibitor first described in cell lines. TP-064 can decrease PRMT4 protein levels as shown in FIG. 10A.

Dosage, delivery of TP-064: PRMT4 specific inhibitor (TP-064, 30 mg/kg/day for 7 days, IP) was administered once daily for 7 or 21 days to young (4-6 months) and aged (9-12 months) C57BL/6J and 3xTg-AD, male and female mice. The inventors have found a dose response curve that evidences that 30 mg/kg (I.P.) of TP-064 decreases PRMT4 (FIG. 10A). TP-064 was dissolved in 100% DMSO, diluted in 0.9% saline (total injection volume=100 μL, ˜1.5% DMSO in mouse), greater than 5% DMSO can affect the brain.

Crossing the BBB with TP-064: The main structure of TP-064 is phenoxybenzamide (TOCRIS). Phenoxybenzamide and associated compounds cross the BBB. This is further supported by the inventors' studies that evidence that TP-064 can lower brain PRMT4 protein levels in a dose-dependent manner (FIG. 10A).

Toxicity: The inventors performed complete blood counts in TP-064 treated (7 days) mice and observed no abnormalities. The inventors have on-going studies to screen for major organ toxicity. Plasma liver enzymes (ALT/AST) and kidney function were normal in TP-064-treated mice.

PRMT4-AAV approach: To demonstrate PRMT4 (also known as CARM1) target specificity, the inventors implemented neuron-specific viral knockdown of PRMT4/CARM1 (AAV/PHP.eB-hSYN1-GFP-U6-m-CARM1-shRNAmir). This viral construct was previously designed and was available to the inventors, as shown in FIGS. 10B-10D. The inventors used the PRMT4-AAV (1×10¹¹ Vector Genomes/kg, retro-orbital injection) as another way to achieve PRMT4 knockdown by a single injection of PRMT4-AAV incubated for 30 days as demonstrated.

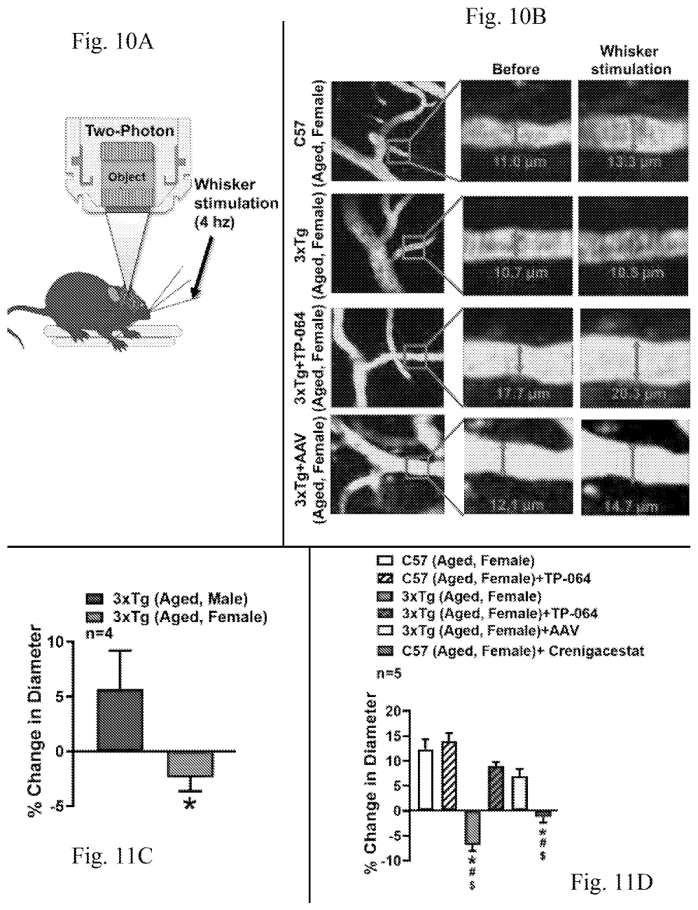

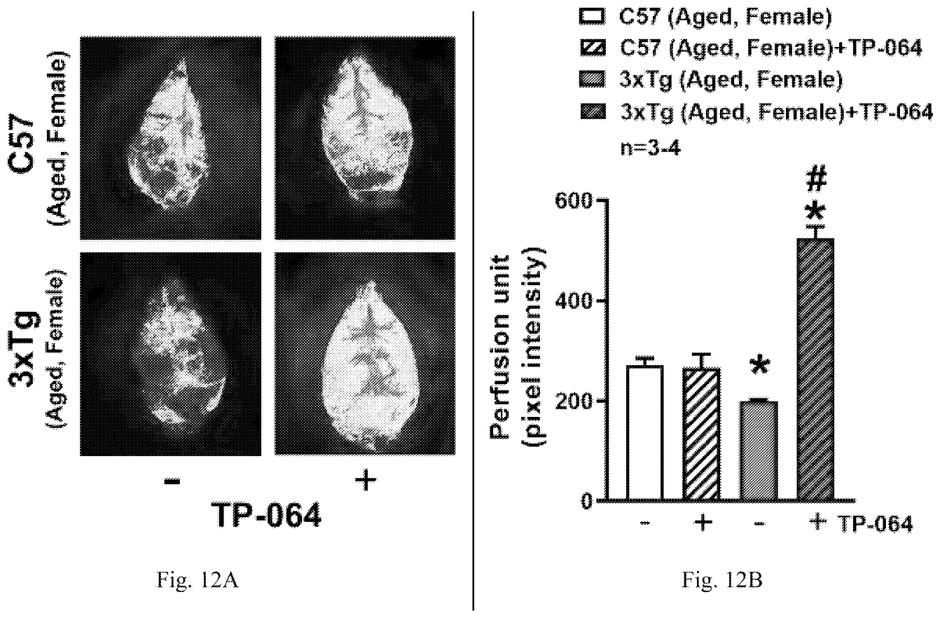

Determining if inhibition of PRMT4 can promote neurovascular coupling. The inventors showed that elevated PRMT4 protein expression in aged 3xTg-AD female mice can depress cerebral blood flow and neurovascular coupling, but was reversed with PRMT4 inhibition (TP-064 or PRMT4-AAV). One reason for this is the loss of neurovascular coupling is associated with the early progression of AD. Whisker-barrel stimulation is a well-established model for examining neurovascular coupling [Rodgers (our in-house collaborator) et al., 2006, 2008, 2009]. The inventors measured cortical microvessel vascular reactivity with two-photon laser scanning microscopy while evoking 30 sec of whisker stimulation in mice. Based on the inventors' findings that aged 3xTg-AD female mice had enhanced PRMT4 expression (FIGS. 2A-3C and 5A-5D), the inventors' results evidence that aged 3xTg-AD female mice presented decreased cortical microvessel diameter as compared to aged 3xTg-AD male mice 30 see (4 Hz) after whisker stimulation. This was reversed (increased cortical microvessel diameter) in TP-064 or PRMT4-AAV-treated aged 3xTg-AD female mice, as shown in FIGS. 11A-11D. Moreover, cortical cerebral blood flow was decreased in aged 3xTg-AD female mice but enhanced by TP-064 via laser speckle contrast imaging, as shown in FIGS. 12A and 12B. Young 3xTg-AD females have decreased PRMT4 protein levels v. aged counterparts (FIG. 2), to suggest that PRMT4 changes with age. However, decreased PRMT4 via TP-064/PRMT4-AAV did not affect vascular diameters or perfusion in C57 (FIGS. 11,12). This could be caused by AD-mediated autoregulation impairment⁸²⁻⁸⁵ or due to the BBB dysfunction (seen in 3xTg-AD mice, and not in C57) (FIG. 11).

Design: Brain Imaging via Two-Photon Laser Scanning Microscopy and Laser Speckle Contrast Imaging. Aged male/female 3xTg-AD or C57BL/6J (control) received TP-064 (30 mg/kg, IP) daily for 7 days (acute endpoint) or 21 days (chronic endpoint). In a separate set of experiments, PRMT4-AAV (single injection, wait 30 days before testing) were used to decrease PRMT4 levels. In vivo two-photon laser scanning microscopy were performed to visualize cerebral blood flow and cortical vessel diameters (pial penetrating arterioles supplying blood to the hippocampus) in young (4-6 months) and aged (9-12 months) C57BL/6J and 3xTg-AD male and female mice at day 0 (before PRMT4 inhibitor) and at day 7, 21 (after TP-064), and day 30 (for PRMT4-AAV). Additionally, laser speckle contrast imaging was used to monitor overall cortical cerebral blood flow to not only corroborate the inventors' two-photon microscopy studies (monitor few vessels), but also to provide a more global view of cerebral blood flow. To evaluate neurovascular coupling, mice received whisker-barrel stimulation while under two-photon laser scanning microscopy or laser speckle contrast imaging for the measurement of vascular reactivity. Mice were sacrificed 7 or 21 days after TP-064 or 30 days after PRMT4-AAV treatment. There are strong relationships between improved cortical cerebral blood flow and improved functional recovery. An advantage of two-photon laser scanning microscopy enables repeated measures of blood vessels within the same animal and localized area of interest.

Results: Aged 3xTg-AD male mice have increased pial microvessel diameter after whisker stimulation, suggesting that neurovascular coupling is intact contrary to aged 3x-Tg AD female mice. Pial microvessel diameters were enhanced in TP-064-treated aged 3xTg-AD female mice. Overall enhancement of cerebral blood flow and microvessel diameters were shown for two-photon laser scanning microscopy and laser speckle contrast imaging (FIGS. 11,12).

Pharmaceutical Compositions. The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents. Exemplary therapeutics include those that reduce PRMT4 protein levels in the patient (including TP-064, the E3 ubiquitin ligase SCF^{FBXO9}, and/or AAV knockdown of PRMT4, for example).

TP-064 is a potent and selective PRMT4 inhibitor with greater that 100-fold selectivity over other histone methyltransferases and non-epigenetic targets. TP-064 inhibits methylation of H3 (1-25) and MED12 in cell. TP-064 has a formula of C₂₈H₃₄N₄O₂ and name of N-Methyl-N-((2-(1-(2-(methylamino)ethyl)piperidin-4-yl)pyridin-4-yl)methyl)-3-phenoxybenzamide.

SCF^{FBXO9} (Skp1-Cullin1-Fbox) is a specific E3 ubiquitin ligase that interacts with PRMT4 via a phosphodegron. The F-box protein in SCF^{FBXO9} is FBX09.

Alzheimer's disease (AD) is the leading cause of mortality, disability, and long-term care burden in the United States, with women comprising the majority of AD diagnoses. While AD-related dementia is associated with tau and amyloid beta accumulation, concurrent derangements in cerebral blood flow have been observed alongside these proteinopathies in humans and rodent models. The homeostatic production of nitric oxide synthases (NOS) becomes uncoupled in AD which leads to decreased NO-mediated vasodilation and oxidative stress via the production of peroxynitrite (ONOO—·) superoxide species. Here, we investigate the role of the novel protein arginine methyltransferase 4 (PRMT4) enzyme function and its downstream product asymmetric dimethyl arginine (ADMA) as it relates to NOS dysregulation and cerebral blood flow in AD. ADMA (Type-1 PRMT product) has been shown to bind NOS as a non-canonic ligand causing enzymatic dysfunction. Our results from RT-qPCR and protein analyses suggest that aged (9-12 months) female mice bearing tau- and amyloid beta-producing transgenic mutations (3xTg-AD) express higher levels of PRMT4 in the hippocampus when compared to age- and sex-matched C57BL6/J mice. In addition, we performed studies to quantify the expression and activity of different NOS isoforms. Furthermore, laser speckle contrast imaging analysis was indicative that 3xTg-AD mice have dysfunctional NOS activity, resulting in reduced production of NO metabolites, enhanced production of free-radical peroxynitrite, and decreased cerebral blood flow. Notably, the aforementioned phenomena can be reversed via pharmacologic PRMT4 inhibition. Together, these findings implicate the potential importance of PRMT4 signaling in the pathogenesis of Alzheimer's-related cerebrovascular derangement.

The inventors sought to investigate differential PRMT expression in AD, which directly influences ADMA production, and affects NOS function as well as cerebral blood flow. Our results suggest that 1) PRMT4 (a type-1 enzyme) was enhanced in 3xTg-AD mice, which 2) enhanced ADMA levels, 3) to dysregulate eNOS and decrease NO production, and 4) decrease cerebral blood flow.

Animal Preparation. All experimental procedures involving animal subjects were approved by the Institutional Animal Care and Use Committee of Louisiana State University Health Sciences Center in Shreveport (Protocol number: P-20-013). Female 9- to 12-month-old 3xTg-AD (Jackson Laboratory, Bar Harbor, ME, cat.#34830-JAX) and 9- to 12-month-old control C57BL/6J (Jackson Laboratory, Bar Harbor, ME) mice were acclimated for 1 week prior to experimentation in animal husbandry facilities. Mice were house in a climate-controlled environment (20°±1° C., humidity 50±5%) under a 12:12-hour light-dark cycle (lights on at 7:00 a.m., 7:00 p.m.) and had access to standard mice chow and water available ad libitum. All experiments consisted of randomized controlled trials. No experiments in this study were pre-registered. Experiments were conducted between 7:00 a.m. to 8:00 p.m. Following experimental endpoints, mice were euthanized by being placed in a small plexiglass chamber and 5% isofluorane (Cat. No. 21295098, Patterson Veterinary, Greeley, CO) vapors were administered until animals were fully sedated and not responsive to sensory stimuli (active paw reflex absent, no eyeblink detected). After sedation was confirmed, mice were decapitated, and tissues were dissected for further analysis. 3xTg-AD mice were developed by Dr. Frank LaFerla, University of California Irvine, donated to Mutant Mouse Resource & Research Centers and sold through Jackson Laboratories. 3xTg-AD mice are homozygous for three mutant alleles (Psen1, APPSwe, tauP301L), and were generated by co-injection of APPSwe and tauP301L transgenes into single cell embryos already bearing the PsenI knock-in mutation. Cross breeding of these mice with mice only bearing PsenI mutation produced homozygous offspring bearing all three mutations.

Treatment regimen: Aged (9-12 month) females from both C57BL6/J as well as 3xTg-AD strains were randomly selected and placed in experimental groups. Treatment consisted of specific PRMT4 inhibitor TP-064 (Cat. No. 6008, Tocris, Minneapolis, MN) (Zhang, de Boer, van der Wel, Van Eck, & Hoekstra, 2021); (Zhong et al., 2018). The pharmacological mechanism of TP-064 has not been well-elucidated though it is thought to act as an allosteric inhibitor of the PRMT4 active site (Nakayama et al., 2018). Based on the pharmacokinetic data provided by the Structural Genomics Consortium (https://www.thesgc.org/), a dose of 30 mg/kg was selected. TP-064 was initially dissolved in dimethyl sulfoxide (DMSO) to a concentration of 30 mg/ml, and then diluted once again 1:3 in sterile saline with a final drug concentration of 10 mg/ml. Following dilution, TP-064 was administered via intraperitoneal (IP) injection once daily for 7 days. Final dose was calculated based on animal weight.

Real-time quantitative PCR: QIAGEN RNeasy Mini Kit was used to extract RNA (Cat. No. 74104, QIAGEN, Hilden, Germany) from mouse brain tissue. RNA purity and quantity was assessed using A260/A280 and A260/A230 ratios via the NanoDrop apparatus (Thermo Fisher Scientific, Waltham, MA). 500 ng total RNA was used in cDNA synthesis reaction using SuperScript™ III Reverse Transcriptase (Cat. No. 18080051, Thermo Fisher Scientific, Waltham, MA) according to the manufacturer's recommendations in a T100™ Thermal Cycler (BioRad, Hercules, CA). CFX96 Real-Time PCR Detection System was used for qPCR analysis. PCR mix consisted of 0.4 μL of cDNA, 10 μL of iQ™ SYBR® Green Supermix (Invitrogen, Carlsbad, CA), 200 nM of each primer, and nuclease free water for 20 μL final volume reaction. Cycling conditions for qPCR amplification were: 95° C. for 3 min, 95° C. for 10 s, 60° C. for 30 s for 40 cycles. All qPCR target genes were normalized against β-actin.

Capillary-phoresis immunoassay via ProteinSimple®: Quantification of all protein targets was achieved using Simple Western analysis in a Jess™ apparatus per the manufacturer's protocol (ProteinSimple, Bio-techne, Minneapolis, MN). Protein was extracted from dissected tissue in an extraction buffer consisting of T-PER® Tissue Protein Extraction Reagent (Cat. No. 78510, Thermo Fischer Scientific, Waltham, MA) and Halt™ Protease Inhibitor Cocktail (Cat. No. 87785, Thermo Fischer Scientific, Waltham, MA). Following homogenization and centrifugation (10,000×g, 5 min, 4° C.), protein concentration was determined in tissue protein supernatant samples using DC Protein Assay (Cat. No. 5000112, Bio-Rad Laboratories, Hercules, California) according to manufacturer instructions. Tissue lysate was then diluted to a final concentration of 1 μg/μL for analysis, then added in a 4:1 (lysate:mix) ratio to a mix containing 40 μM fluorescent molecular weight marker and dithiothreitol (DTT). Samples were then denatured for 5 min at 95° C. before loading into microwell plate. Antibody diluent (ProteinSimple, Bio-techne, Minneapolis, MN) was used for all antibody dilutions as follows: PRMT4 (1:50, RRID: AB_2068436), PRMT6 (1:100, Cell Signaling Technology cat. 14641), eNOS (1:200, RRID: AB_10850618), nNOS (1:100, RRID: AB_2152488), iNOS (1:100, RRID: AB_10000625), DDAH2 (1:50, RRID: AB_2640442) Antibodies were detected using HRP-conjugated secondary anti-rabbit, and -mouse. Relative protein levels were calculated by measuring the area under the peak of a chemiluminescent chromatogram and normalized to total capillary chemiluminescence for a given sample. All analyses were carried out using Compass for SW software (version 6.0.0, Protein Simple, Bio-techne, Minneapolis, MN). Pseudo-blot images are computer generated based on peak values derived from the Compass for SW software.

Nitrate/Nitrite colorimetric assay: Following euthanasia, whole brains were dissected from mice. Cold (4ºC) phosphate-buffered saline (PBS) and glass tissue grinders were used to homogenize the whole brain. Homogenates were centrifuged at 10,000×g for 20 min at 4° C. The supernatant was collected and filtered using 10 kDa ultrafilter (Cat. No. MRCPRT010, Millipore, Burlington, VA). Nitrate and nitrite were quantified per manufacturer's instructions (Cat. No. 780001, Cayman Chemical, Ann Arbor, MI). In summary, 80 μL of supernatant was loaded into a 96-well plate with 10 μL enzyme cofactor, and 10 μL nitrate reductase. Following 3 hrs of incubation, 100 μL Griess reagent was added, and plate was incubated for 30 min. The standard curve was provided by the manufacturer, and all sample concentrations were determined from the standard curve values. Changes in absorbance were measured at 550 nm. Final nitrate/nitrite levels were expressed in μM concentration.

Asymmetric dimethyl arginine ELISA: Cardiac blood (500 μL) was collected in 0.4 M EDTA-coated tubes (Cat. No. E5134, Sigma, Burlington, MA) and left at room temperature for 15 min. Blood samples were centrifuged for 10 min at 20° C., 1500×g. The serum was collected in a clean polypropylene tube and stored at 4° C. until needed. Asymmetric dimethyl arginine levels were quantified using ADMA direct (mouse/rat) ELISA kit following the manufacturer instructions (Cat. No. ALX-850-327-KI01, Enzo Life Sciences, Farmingdale, NY). Briefly, blood serum samples were brought to room temperature (20)° C. and plated with 50 μL ADMA antibody into a 96-well plate. The plate was incubated overnight at 4° C. Following incubation, wells were washed 5 times using assay wash buffer, and 100 μL of conjugate was added to each well. Plate was incubated at room temperature (20° C.) for 1 hr on a horizontal shaker. After incubation, plate was washed 5 times using assay wash buffer, and 100 μL of substrate was added. Plate was incubated at room temperature (20)° C. in the dark for 10 min, after which stop solution was added to each well. The standard curve was provided by the manufacturer, and all sample concentrations were determined from the standard curve values. Changes in absorbance were measured at 405 nm against 620 nm. Final ADMA levels were expressed in μM concentration.

OxiSelect™ 3-Nitrotyrosine ELISA: Protein was extracted from dissected tissue in an extraction buffer consisting of T-PER® Tissue Protein Extraction Reagent (Cat. No. 78510, Thermo Fischer Scientific, Waltham, MA) and Halt™ Protease Inhibitor Cocktail (Cat. No. 87785, Thermo Fischer Scientific, Waltham, MA). Following homogenization and centrifugation (10,000×g, 5 min, 4° C.), protein concentration was determined in the supernatant using DC Protein Assay (Cat. No. 5000112, Bio-Rad Laboratories, Hercules, California) according to manufacturer instructions. Levels of 3-Nitrotyrosine were determined using OxiSelect™ 3-Nitrotyrosine ELISA (Cat. No. STA-305, Cell Biolabs Inc., San Diego, CA). Briefly, 50 μL of protein lysate was added to a pre-coated 96-well plate and incubated at room temperature (20° C.) for 10 min on an orbital shaker. 50 μL of anti-nitrotyrosine antibody (1:1000, Cell Biolabs Inc., San Diego, CA) was added to each well and plate was incubated for 1 hr at 20° C. Wells were washed 3 times using assay wash buffer, and 100 μL of secondary antibody-enzyme conjugate (1:1000, Cell Biolabs Inc., San Diego, CA) was added to each well. Plate was then incubated for 1 hr at 20° C. on an orbital shaker. Following incubation, wells were washed 3 times using assay wash buffer. 100 μL warmed substrate solution was added to each well, at 10 min intervals. Subsequently, 100 μL of the stop solution was added to each well. The standard curve was provided by the manufacturer, and all sample concentrations were determined from the standard curve values. Changes in absorbance were measured at 450 nm. Final 3-Nitrotyrosine levels were expressed in μM concentration.

Surgical Preparation and Anesthetic Procedure: Mice were anesthetized with 4% isoflurane (Cat. No. 21295098, Patterson Veterinary, Greeley, CO) and a mixture of O₂ and N₂O (30:70) via mask. Hair was shaved at the site of surgery (from the nape up to the eyes). Body temperature was maintained at 37° C. by a rodent heating pad with anal probe as a temperature reference (ThermoStar, RWD Life Science, San Diego, CA). Anesthesia was maintained by lowering isoflurane from 4% to 1%.

Thinned-skull preparation: Mice were placed in a stereotaxic frame to stabilize the skull and artificial tears (Patterson Veterinary, Greeley, CO) was applied in each eye. Dissection scissors and forceps were used to reveal the skull. A high-speed dental drill (RWD Life Science, San Diego, CA) was used to thin the skull creating a small window (1 mm in diameter) approximately 2 mm rostral and 1 mm lateral to the lambda, with saline irrigation to prevent overheating of the skull tissue. Laser speckle contrast imaging or two-photon laser scanning microscopy analyses were conducted following this preparation.

Laser speckle contrast imaging: Cerebral microvasculature was imaged by utilizing the thin skull preparation method. After thinning the skull, the mouse was placed in a stereotaxic apparatus. The RFLSI III Speckle Contrast Imaging System (RWD Life Science, San Diego, CA) was used to conduct laser speckle contrast imaging experiment. Mice were imaged for a 5 min time span to calculate average flow. Wavelength was scaled 0-750 nm. LCSI software (VERSION, RWD Life Science, San Diego, CA) was used to calculate perfusion intensity.

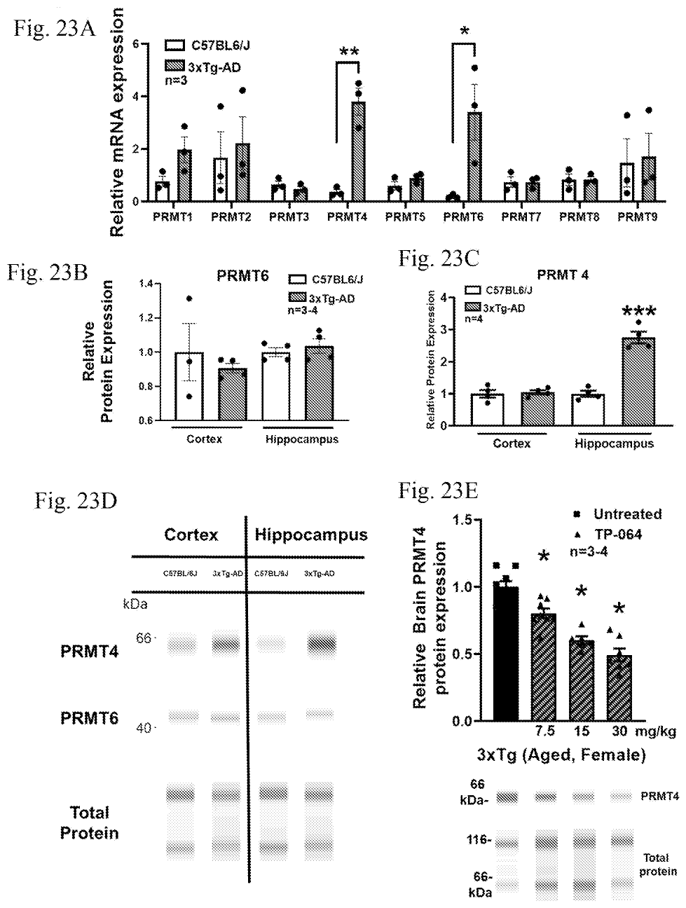

Results: Aged female 3xTg-AD mice exhibit differential expression of PRMT4 which can be reversed by TP-064. Quantitative real-time qPCR of mRNA derived from whole-brain lysates indicates that aged female 3xTg-AD mice (9-12 months) exhibit significantly increased expression of both PRMT4 [343.7±52.3% (p 0.01)] and PRMT6 [550.3±90.9% (p 0.02)] mRNA (for information regarding primer sequences, see Table 1) (FIG. 23A). Age/sex-matched C57BL/6J mice did not show significant differences in PRMT6 protein expression in cortex [−9.3±14.4%, (p=0.544)] or hippocampus (3.6±5.0%, p=0.500). Additionally, no significant changes were observed in cortical PRMT4 protein expression of 3xTg-AD mice as compared to age-/sex-matched C57BL/6J mice [4.59±13.7% (p=0.759)]. However, hippocampal PRMT4 protein expression was significantly enhanced in aged female 3xTg-AD mice as compared to age- and sex-matched C57BL/6J mice [175±20.6% (p<0.0001)] (FIGS. 23B and 23C). Treatment with specific PRMT4 inhibitor (TP-064, 30 mg/kg, 7 days, IP) reduced PRMT4 expression in whole brain lysate in a dose-dependent manner in treated 3xTg-AD groups relative to untreated 3xTg-AD samples at 7.5 mg/kg [p=0.0063], 15 mg/kg [p<0.0001], and 30 mg/kg [p<0.0001] via one-way ANOVA (F(3,25)=31.51, p<0.0001) with Bonferroni post-hoc analysis (FIG. 23D).

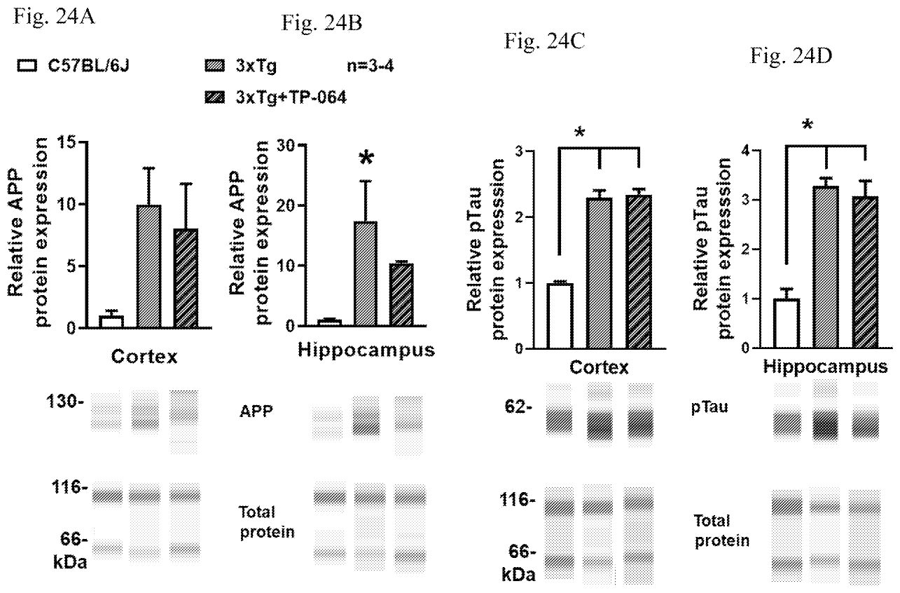

Aged female 3xTg mice have higher levels of brain amyloid precursor protein and phosphorylated-tau compared to age- and sex-matched C57 mice. Relative protein levels for amyloid precursor protein (APP) as well as phosphorylated tau (pTau) were measured using capillary-phoresis. One-way ANOVA with Tukey's post-hoc analysis comparing APP levels [F(3,11)-4.233, p=0.0322] in cortical and hippocampal lysates revealed significantly enhanced APP in the hippocampus of 3xTg mice [1741%±6.62% (p=0.0298)] compared to controls [100%±19.7%]. APP levels were increased in cortical lysates of 3xTg-AD mice [996%±29.4%] compared to control animals [100%±41.7%] (FIG. 24A). APP levels were not significantly changed in the hippocampus and cortex 3xTg-AD mice treated with TP-064 [1040%±31.1%; 801%±36.3%]. One-way ANOVA with Tukey's post-hoc analysis comparing pTau levels [F(2,6)=84.7, p<0.0001] in cortical and hippocampal lysates revealed significantly enhanced pTau in both the hippocampus and cortex of 3xTg mice [329%=15.3% (p=0.001); 230%±11.4% (p<0.0001.)] as well as their TP-64-treated counterparts [307%±31.4% (p=0.0017); 234%±8.33% (p<0.0001)] compared to Bcontrols [100%±19.5%; 100%±2.52%] (FIG. 24b).

Aged female 3xTg-AD mice have increased levels of asymmetric dimethylarginine (ADMA), enhanced expression of dimethylarginine dimethylaminohydrolase 2 (DDAH2), and increased levels of peroxynitrite derivative 3-nitrotyrosine.

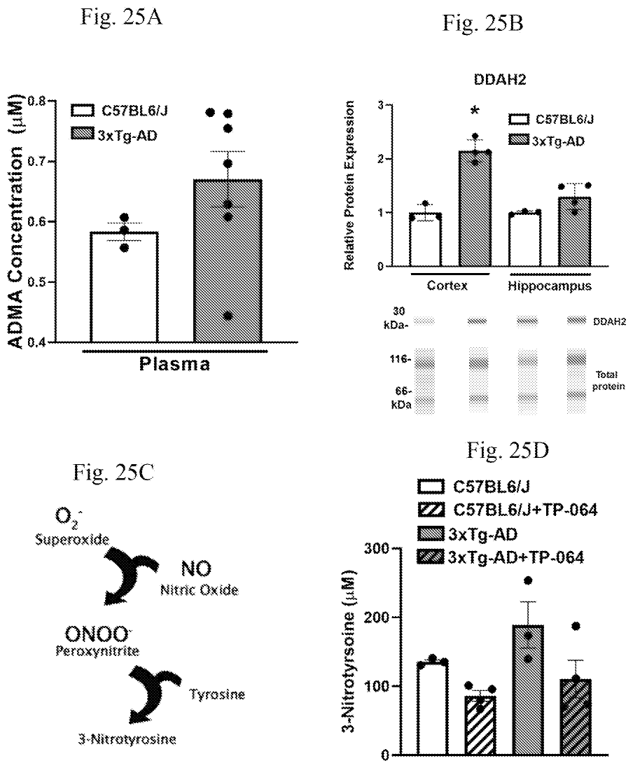

The primary product of PRMT4 (type I PRMT) is ADMA. We determined ADMA levels in plasma samples from aged female 3xTg-AD mice and age-/sex-matched C57BL/6J mice. Plasma levels of ADMA were elevated in 3xTg-AD mice [0.670±0.0460 μM] as compared C57BL/6J [0.583±0.0145 μM (p=269)] (FIG. 25A). Protein levels of DDAH2 (Zhu et al., 2019) (primary ADMA scavenger) were quantified via capillary-phoresis. DDAH2 protein levels were significantly enhanced in 3xTg-AD cortex [150.0±14.2% (p=0.0005)] as compared to C57BL6/J controls, however no significant changes were observed in the hippocampus [29.8±14.4% (p=0.0927)] (FIG. 25B). Production of peroxynitrite radicals is indicative of NOS dysfunction. Stable peroxynitrite derivative 3-nitrotyrosine was quantified via ELISA. Two-way ANOVA with Tukey's post hoc analysis [F(1,10)=3.18, p=0.105] revealed no significant changes, however aged female 3xTg-AD mice [189±33.7 μM] exhibited elevated levels of 3-nitrotrosine when compared to age-/sex-matched C57BL/6J mice [136±2.95 μM (p=0.413)] (FIG. 25d). Administration of TP-064 reduced 3-nitrotyrosine levels in both 3xTg-AD [110±27.2 μM (p=0.115)] as well as C57BL/6J mice [86.9±7.71 μM (p=0.422)] compared to their untreated counterparts, respectively (FIG. 25d).

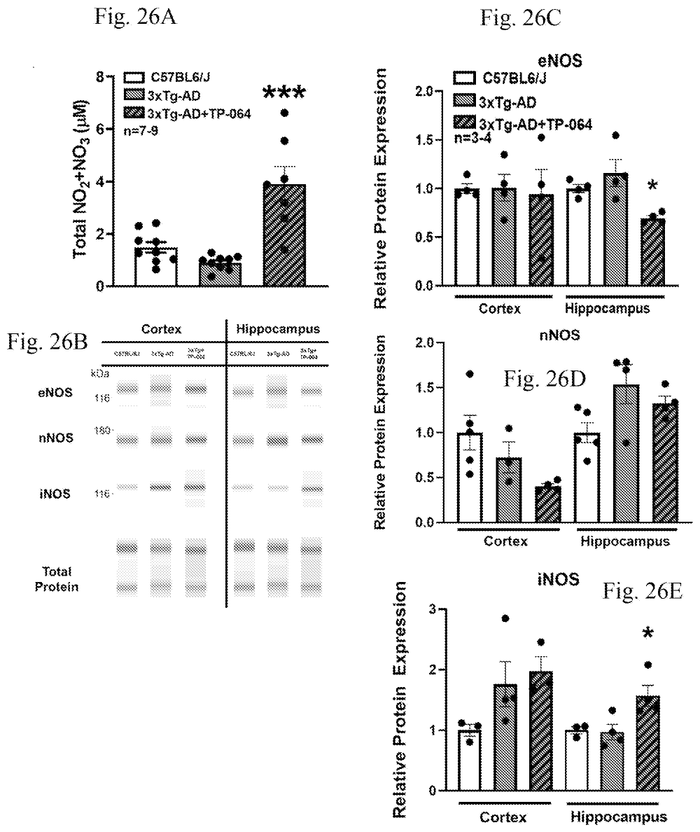

Nitric oxide synthase function was impaired in aged female 3xTg-AD mice. Plasma ADMA has been shown to inhibit nitric oxide synthase activity (McCarty, 2004); (Jiang et al., 2006). Nitric oxide metabolites nitrate (NO₃) and nitrite (NO₂) were quantified using Griess reaction. The level of total NO₃+NO₂ in whole-brain lysate was lower in aged female 3xTg-AD mice [0.895±0.0936 μM] as compared to age-/sex-matched C57BL/6J mice [1.48±0.603 μM], TP-064 administration [3.90±0.667 μM] (30 mg/kg, IP, 7 days) significantly elevated NO₃±NO₂ levels in 3xTg-AD animals as compared to C57BL6/J [p=0.0003] as well as untreated 3xTg-AD [p<0.0001] mice as determined via one-way ANOVA with Tukey's post hoc analysis [F(2,22)=19.15, p<0.0001] (FIG. 26A).

Protein analyses of endothelial (eNOS), as well as neuronal (nNOS) and inducible (iNOS) nitric oxide synthases were conducted using capillary-phoresis. One-way ANOVA with Tukey's post-hoc analysis comparing eNOS levels [F(2,9)=0.0465, p=0.9548] in cortical lysates revealed no significant differences between control [100%±4.93% (p=0.999)], untreated 3xTg-AD mice [101%±13.9% (p=0.966)], and treated 3xTg-AD mice[94.0%±25.6% (p=0.959)], however significant reduction in eNOS protein was observed in analysis [F(2,9)-7.65, p=0.0114] of hippocampal lysates between untreated[116%±13.9%] and treated 3xTg-AD mice [69.5%±2.71%], [p=0.00990], no changes were observed in hippocampal lysates in control mice [100%±4.13%, (p=0.423)] (FIG. 26C). One-way ANOVA with Tukey's post-hoc analysis comparing nNOS levels [F(2,9)-3.83, p=0.0625] in cortical lysates revealed no significant differences between control [100%±19.3% (p=0.496)], untreated 3xTg-AD mice[72.4%±17.3% (p=0.0519)], and treated 3xTg-AD mice [40.3%±2.88% (p=0.426)], additionally no significant differences in nNOS protein were observed in analysis [F(2,10)=3.70, p=0.0626] of hippocampal lysates between control [100%±11.2%, (p=0.0555)] untreated [154%±21.8%, (p=0.280)] and treated 3xTg-AD mice[133%±8.15%, (p=0.595)] (FIG. 26D). One-way ANOVA with Tukey's post-hoc analysis comparing iNOS levels [F(2,7)-2.70, p=0.135] in cortical lysates revealed no significant differences between control [100%±9.88% (p=0.229)], untreated 3xTg-AD mice [176%±37.3% (p=0.141)], and treated 3xTg-AD mice [197%±24.1% (p=0.867)]. However, significant increase in iNOS protein was observed in analysis [F(2,8)-5.99, p=0.0257] of hippocampal lysates between untreated [97.0%±12.7%] and treated 3xTg-AD mice [157%±17.5%], [p=0.0335], no changes were observed in hippocampal lysates in control mice [100%±6.07%, (p=0.987)] (FIG. 26D).

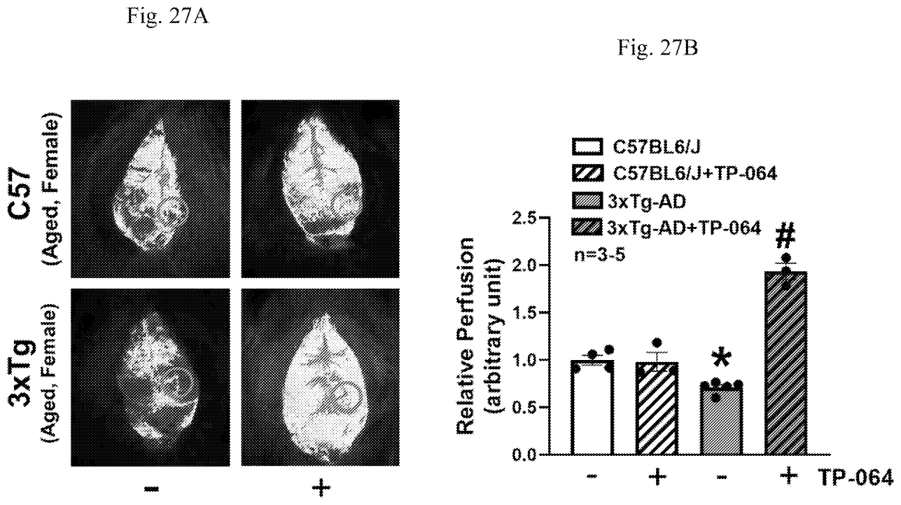

PRMT4 inhibition via TP-064 enhanced regional cortical cerebral blood flow. Loss of NO-derived vasodilation results in chronic hypoperfusion in AD. Regional cortical CBF was measured via laser speckle contrast imaging (LSCI). Two-way ANOVA with Tukey's post hoc analysis [F(1,11)=100.3, p<0.0001] revealed regional cortical CBF was significantly reduced in aged, female 3xTg-AD [71.2%±2.82%] mice when compared with C57BL/6J [100%±5.05%] [p=0.0167]. Administration of PRMT4 specific inhibitor TP-064 (30 mg/kg, IP, 7 days) significantly enhanced regional cortical CBF in 3xTg-AD mice [194%±8.6%] as compared to untreated 3xTg-AD mice [71.2%±2.82%, (p<0.0001)], treated and untreated C57BL/67 mice [100%±5.05% (p<0.0001); 98.3±10.1%, (p<0.0001)], respectively (FIG. 27).

Discussion: In this study, we established that female 3xTg-AD mice have 1) elevated levels of hippocampal PRMT4 protein expression, 2) higher serum levels of ADMA, 3) elevated peroxynitrite derivative 3-nitrotyrosine production, 4) impaired NO metabolite production, and 5) reduced cortical regional cerebral blood flow when compared to age and sex-matched controls. Additionally, treatment with specific PRMT4 inhibitor TP-064 A) reduced PRMT4 expression in the brain in a dose-dependent manner, B) reduced levels of 3-nitrotyorosine, C) significantly enhanced production of NO metabolites, and D) restored regional cortical cerebral blood flow. These results suggest the pathological role of PRMT4 signaling and its influence over cerebral blood flow in Alzheimer's disease.

Alzheimer's disease (AD) is a neurodegenerative disorder that progresses insidiously, and ultimately results in cognitive, learning and memory deficits. These deficiencies have devastating functional consequences, and with no known cure for AD, victims of the disease often require 24-hour care for the remainder of their lives (Eratne et al., 2018). Women are more at risk than men to develop AD. A hallmark of neurodegeneration and cerebral atrophy seen in Alzheimer's patients has been attributed to the inherent neurotoxicity evoked by aggregates of amyloid beta (AB) and microtubule-associated protein tau (MAPT). However, derangements in cerebral blood flow (CBF) are a crucial pathogenic factor in AD. Enhancing cerebral blood flow in APP/PS1 mice leads to improvements in cognition, even in mice as old as 16 months. In the current study, 3xTg-AD mice exhibit cerebral blood flow impairment when compared to age- and sex-matched controls (FIG. 27). Conversely, in patient populations, there is a clear correlation between hypertension and the development of AD, as hypertension is a known risk factor for AD. Restoration of cerebral blood flow is a modifiable risk factor that offers an avenue for pharmacological targets that can improve the functional outcomes of Alzheimer's patients. Our data indicate that administration of TP-064, a specific inhibitor for type-1 protein arginine methyltransferase 4 (PRMT4) robustly increased CBF in treated 3xTg-AD mice (FIG. 27). The relationship between regional cortical cerebral blood flow and its impact on deeper brains structures is shown in studies in which reduced regional cerebral blood flow were associated with decreased cognition, indicating that regional CBF impacts the CBF and functionality of deeper, more sensitive brain structures (i.e. hippocampus).

Protein arginine methyltransferases (PRMTs) are a novel class of enzymes that catalyze the transfer of methyl groups from s-adenosylmethionine to arginine residues. It is thought that PRMTs are mediators of genetic expression that determine DNA-histone binding patterns to alter gene expression. There are three subtypes of PRMT enzymes, each of which produce different methylation patterns. Type 1 PRMTs (1,2,3,4,6,8) produce asymmetrical dimethylarginine (ADMA), type 2 PRMTs (5,9) produce symmetric dimethylarginine (SDMA), and the lone type 3 PRMT (7) produces monomethyl arginine (MMA). Despite being a common posttranslational modification, arginine methylation is under-investigated as compared to posttranslational modifications such as phosphorylation or acetylation. Arginine methylation has been shown to have a role in neurodegenerative pathologies such as amyotrophic lateral sclerosis and Huntington's disease. Additionally, arginine methylation by PRMT4 has been associated with changes in cell senescence associated with aging. In this study, our RT-qPCR panel identified both PRMT4 as well as PRMT6 as being significantly enhanced in the brain of 3xTg-AD mice when compared to control animals (FIG. 23A). Further protein-level quantification revealed specifically that PRMT4 protein was significantly enhanced in hippocampal lysates of 3xTg-AD mice, which focused our investigation on PRMT4 (FIGS. 23B and 23C). The expression of PRMT4 could be reduced in a dose-dependent manner with the administration of TP-064 (FIG. 23D). Others have reported that PRMT 4 is expressed in the central nervous system (Ishino, Shimizu, Tohyama, & Miyata, 2022), and as PRMT4 is a type-1 PRMT, it produces ADMA. Our findings suggest that ADMA production is enhanced in the serum of 3xTg-AD mice (FIG. 25B). ADMA can be scavenged from the circulation, and subsequently degraded by dimethylarginine dimethylaminohydrolase-2 (DDAH2). We have shown that DDAH2 protein is elevated in both cortical and hippocampal lysates in 3xTg-AD animals, which may be a compensatory response to increased ADMA production which was also observed in 3xTg-AD animals (FIG. 25A). The most well-known function of PRMT4 is the methylation of histone H3. The role of PRMT 4 in the central nervous system is primarily involved in the development of astroglial and oligodendritic cells. Additionally, PRMT 4 has been shown to mediate the development of glial cells in early development of the CNS, and the loss of PRMT4 is embryonically lethal. The inventors' findings evidence that PRMT expression patterns are linked to pathological conditions.

The nitric oxide synthase (NOS) family of enzymes are the primary source of nitric oxide (NO) in the body, making them crucial mediators of vascular tone as NO is a vasodilatory gasotransmitter (Kelly & Smith, 1996). The canonic ligand of NOS enzymes is L-arginine, and in the NOS active site, L-arginine is converted to L-citrulline, and the NO byproduct is produced. The phrase “NOS uncoupling” is used to describe a state of NOS function in which the enzyme expression is not affected, though peroxynitrite (ONOO·—) radical production is enhanced while NO production is decreased. Our results suggest that aged female 3xTgAD mice have higher levels of peroxynitrite derivative 3-nitrotyrosine as well as higher levels of ADMA production despite having no significant change in protein expression across all NOS isoforms when compared to the control group, which is indicative of NOS uncoupling (ie. a functional change in NO production that is unrelated to the relative amount of NOS expression) (FIGS. 24A and 25C—25E). Notably, treatment with PRMT4 inhibitor caused nonsignificant reduction in both eNOS and nNOS in the brain (FIGS. 25C and 25D). This is potentially due to the restoration of NOS function that was seen in the enhancement of NOS metabolites in FIG. 26A. As NOS functionality was restored, there was a reduced demand for enzyme production which was increased in untreated 3xTg animals, to possibly compensate for the uncoupled NOS enzymes. The enhancement in iNOS levels seen with treatment of TP-064 may possibly be linked to a protective response seen in reperfusion injury models, as the inhibition of iNOS expression was shown to be detrimental to tissue recovery and iNOS expression has been shown to be regulated by hypoxia. Additionally, treatment with PRMT4 inhibitor TP-064 reversed the production of 3-nitrotyrosine in 3xTg-AD animals, as well as restored the production of NO metabolites (FIGS. 24D and 25A). Loss of NO signaling has been shown to cause cerebral hypoperfusion (Park et al., 2020). The inventors' finding of reduced regional cortical cerebral blood flow in 3xTg-AD animals corroborates these results, and administration of PRMT4 inhibitor TP-064 results in significantly increased regional cortical cerebral blood flow in 3xTg-AD animals (FIG. 27). Considering this finding in the context of our NOS expression studies hippocampal eNOS protein expression may be downregulated in the treated 3xTg-AD group as a response to the sharp increase in CBF. NOS uncoupling has also been shown to be a contributing factor in the pathogenesis of AD. 3xTg-AD mice exhibited higher levels of amyloid precursor protein (APP) and phosphorylated tau (pTAU) in the hippocampus and cortex compared to control animals, though administration of TP-064 had no significant effect on expression of these proteins in 3xTg mice in either tissue type (FIG. 24). This is likely due to the relatively short duration of the dosage paradigm in these studies. While the site and specific mechanism of NOS uncoupling is unknown, the binding of type-1 PRMT product ADMA to NOS is speculated to contribute to NOS uncoupling by increasing the spin state of the heme-bound oxygen in the active site of NOS, which increases the likelihood of improper electron transfer. Interestingly, our findings suggest that the ADMA scavenger DDAH2 is upregulated in 3xTg-AD mice, however this may be a compensatory mechanism which may be activated in response to 3xTg-AD mice having higher levels of ADMA (FIGS. 25A and 25B).

Conclusion: Overall, the inhibition of PRMT4 via TP-064 resulted in improvements in the functional measurements of NOS uncoupling (reduced 3-nitrotyrosine (FIG. 26D), enhanced NO metabolite production (FIG. 25A) as well as restored CBF in 3xTg-AD mice (FIG. 26), these findings demonstrate that type-1 PRMT4 activity acutely influences CBF and NOS function in AD pathology.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration. The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings. The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration. Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery. Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992). Oral transmucosal absorption is generally rapid because of the rich vascular supply to the mucosa and allows for a rapid rise in blood concentrations of the therapeutic. For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices.

Formulations for sublingual administration can also be used, including powders and aerosol formulations. Exemplary formulations include rapidly disintegrating tablets and liquid-filled soft gelatin capsules.

The pharmaceutical compositions of the invention may be dispensed to the subject under treatment with the help of an applicator. The applicator to be used may depend on the specific medical condition being treated, amount and physical status of the pharmaceutical composition, and choice of those skilled in the art. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the skin surface, liquid in the container is transferred to the skin in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

The pharmaceutical compositions of the invention may be provided to the subject or the medical professional in charge of dispensing the composition to the subject, along with instructional material. The instructional material includes a publication, a recording, a diagram, or any other medium of expression, which may be used to communicate the usefulness of the composition and/or compound used in the practice of the invention in a kit. The instructional material of the kit may, for example, be affixed to a container that contains the compound and/or composition used in the practice of the invention or shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Other routes of administration to the affected area which are contemplated include: transdermal, mucosal, rectal, and vaginal, or topical (for example, in a carrier vehicle, a topical control release patch, in a wound dressing, a hydrocolloid, a foam, or a hydrogel, a cream, a gel, a lotion, an ointment, a liquid crystal emulsion (LCE), and/or a micro-emulsion). An appropriate biological carrier or pharmaceutically acceptable excipient may be used. Compounds administered may, in various embodiments, be racemic, isomerically purified, or isomerically pure.

Topical Formulations: Topical formulations may be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

Transdermal Administration: Transdermal compound administration, which is known to one skilled in the art, involves the delivery of pharmaceutical compounds via percutaneous passage of the compound into the systemic circulation of the patient. Topical administration may also involve the use of transdermal administration such as transdermal patches or iontophoresis devices. Other components may be incorporated into the transdermal patches as well. For example, compositions and/or transdermal patches may be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions may include creams, sprays, lotions, gels, ointments, eye drops, nose drops, ear drops, and the like. In such dosage forms, the compositions of the invention may be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions may contain polyethylene glycol 400. They may be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, may be impregnated with the compositions in solution, lotion, cream, ointment or other such form may also be used for topical application. The compositions may also be applied topically using a transdermal system, such as one of an acrylic-based polymer adhesive with a resinous crosslinking agent impregnated with the composition and laminated to an impermeable backing.

Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir that, in this case, may be either a polymeric matrix as described above, or be a liquid or hydrogel reservoir, or take some other form.

Additional Administration Forms. Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Application Nos. 20030147952, 20030104062, 20030104053, 20030044466, 20030039688, and 20020051820. Additional dosage forms of this invention also include dosage forms as described in PCT Application Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757, such forms incorporated by reference.

Solutions: After a PRMT4 inhibitor has been selected, it may be dissolved into a solution. The solution may be an aqueous-based solution, such as water, saline, or the like. In some variations, other fluids and solutions may be appropriate.

Various formulations of saline are known in the art and may be used with the present invention. For example, the saline may be lactated Ringer's solution, acetated Ringer's solution, phosphate buffered saline (PBS), Dulbecco's phosphate buffered saline (D-PBS), Tris-buffered saline (TBS), Hank's balanced salt solution (HBSS), or Standard saline citrate (SSC).

The saline solutions of the present invention are, in certain embodiments, “normal saline” (i.e., a solution of about 0.9% w/v of NaCl). Normal saline has a slightly higher degree of osmolality compared to blood; however, in various embodiments, the saline may be isotonic in the body of a subject such as a human patient. In certain embodiments, “half-normal saline” (i.e., about 0.45% NaCl) or “quarter-normal saline” (i.e., about 0.22% NaCl) may be used with the present invention. Optionally, about 5% dextrose or about 4.5 g/dL of glucose may be included in the saline. In various embodiments, one or more salt, buffer, amino acid and/or antimicrobial agent may be included in the saline.

In various embodiments, a preservative or stabilizer may be included in the composition or solution. For example, the prevention of the action of microorganisms may be brought about by preservatives such as various antibacterial and antifungal agents, including but not limited to parabens (for example, methylparabens, propylparabens), chlorobutanol, phenol, sorbic acid, EDTA, metabisulfite, benzyl alcohol, thimerosal or combinations thereof. Agents that may be included suitable for use include sterile aqueous solutions or dispersions and sterile powders for the extemporaneous preparation of sterile solutions or dispersions (U.S. Pat. No. 5,466,468, specifically incorporated herein by reference in its entirety). In all cases the composition is preferably sterile and must be fluid to facilitate easy injectability. Solutions are preferably stable under the conditions of manufacture and storage and must be preserved against the contaminating action of microorganisms, such as bacteria and fungi. Examples of stabilizers which may be included include buffers, amino acids such as glycine and lysine, carbohydrates such as dextrose, mannose, galactose, fructose, lactose, sucrose, maltose, sorbitol, mannitol, and the like. Appropriate stabilizers or preservatives may be selected according to the route of administration desired. A particle filter or microbe filter may be used, and may be necessary according to the route of administration desired.

The weight ranges of compounds in the solution may vary. For example, in various embodiments, the composition may comprise about 0.1-10 wt %, more preferably 1-5 wt % PRMT4 inhibitor, about 1-5 wt % preservative/stabilizer, about 1-5 wt % NaCl, and about 85%-97% water. The ratio of PRMT4 inhibitor to water may be varied as needed to achieve the desired treatment of the AD or pre-AD condition.

The solution and/or composition may also be sterilized prior to administration. Methods for sterilization are well known in the art and include heating, boiling, pressurizing, filtering, exposure to a sanitizing chemical (for example, chlorination followed by dechlorination or removal of chlorine from solution), aeration, autoclaving, and the like.

The PRMT4 inhibitor may be formulated into a solution in any number of ways. For example, it may be solubilized by agitation or by sonication, or other methods known in the art. After the PRMT4 inhibitor has been solubilized, it may be administered to a subject in need of treatment of AD or pre-AD condition. In certain embodiments, a PRMT4 inhibitor is admixed with a solution in a closed vacuum container, and the combined solutions are then mechanically agitated for 3-5 minutes and held in a thermo-neutral sonicator until use.

In certain embodiments, solutions of the present invention may be a component of an emulsion, such as a water-in-oil or an oil-in-water emulsion, including a lipid emulsion, such as a soybean oil emulsion. Certain emulsions have been described previously for intravenous (da Silva Telles, et al., 2004, Rev. Bras. Anaestesiol Campianas 54(5):2004) or epidural administration (Chai et al. 2008, British J Anesthesia 100:109-115), such described emulsion techniques incorporated by reference herein.

Pharmaceutical compositions of the present invention comprise an effective amount of one or more PRMT4 inhibitors dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one PRMT4 inhibitor in solution or additional active ingredient will be known to those of skill in the art in light of the present disclosure, as exemplified by “Remington: The Science and Practice of Pharmacy,” 20th Edition (2000), which is incorporated herein by reference in its entirety. Moreover, for animal (for example, human) administration, it will be understood that preparations should meet sterility, pyrogenicity, general safety and purity standards as required by FDA Office of Biological Standards.

In various embodiments, the compositions of the present invention further comprise cyclodextrin. Cyclodextrins are a general class of molecules composed of glucose units connected to form a series of oligosaccaride rings (See Challa et al., 2005, AAPS PharmSciTech 6:E329-E357). In nature, the enzymatic digestion of starch by cyclodextrin glycosyltransferase (CGTase) produces a mixture of cyclodextrins comprised of 6, 7 and 8 anhydroglucose units in the ring structure (α-, β-, and γ-cyclodextrin, respectively). Commercially, cyclodextrins are also produced from starch, but different, more specific enzymes are used. Cyclodextrins have been employed in formulations to facilitate the delivery of cisapride, chloramphenicol, dexamethasone, dextromethoraphan, diphenhydramine, hydrocortisone, itraconazole, and nitroglycerin (Welliver and McDonough, 2007, Sci World J, 7:364-371). In various embodiments, the cyclodextrin of the invention is hydroxypropyl-Beta-cyclodextrin, sulfobutylether-beta-cyclodextrin, alpha-dextrin or combinations thereof. In certain embodiments, cyclodextrin may be used as a solubilizing agent.

In various other embodiments, compositions of the present invention may comprise human serum albumin purified from plasma, or recombinant human serum albumin. In certain embodiments, human serum albumin may be used as a solubilizing agent. In other embodiments, the compositions of the invention may comprise propylene glycol. In other embodiments, the compositions of the invention may comprise perfluorooctyl bromide. In other embodiments, the compositions of the invention may comprise perfluorocarbon. In certain embodiments, perfluorocarbon may be used as a solubilizing agent.

In various embodiments, a preservative or stabilizer may be included in the composition or solution. For example, the prevention of the action of microorganisms may be brought about by preservatives such as various antibacterial and antifungal agents, including but not limited to parabens (for example, methylparabens, propylparabens), chlorobutanol, phenol, sorbic acid, EDTA, metabisulfite, benzyl alcohol, thimerosal or combinations thereof. Agents which may be included suitable for use include sterile aqueous solutions or dispersions and sterile powders for the extemporaneous preparation of sterile solutions or dispersions (U.S. Pat. No. 5,466,468, specifically incorporated herein by reference in its entirety). In all cases the composition is preferably sterile and must be fluid to facilitate easy injectability. Solutions are preferably stable under the conditions of manufacture and storage and must be preserved against the contaminating action of microorganisms, such as bacteria and fungi. Examples of stabilizers which may be included include buffers, amino acids such as glycine and lysine, carbohydrates such as dextrose, mannose, galactose, fructose, lactose, sucrose, maltose, sorbitol, mannitol, etc. Appropriate stabilizers or preservatives may be selected according to the route of administration desired. A particle filter or microbe filter may be used and may be necessary according to the route of administration desired.

Administration of the disclosed compositions in a method of treatment may be achieved in a number of different ways, using methods known in the art. Such methods include, but are not limited to, topically administering solutions, suspensions, creams, pastes, oils, lotions, gels, foam, hydrogel, ointment, liposomes, emulsions, liquid crystal emulsions, and nano-emulsions.

The therapeutic and prophylactic methods of the invention thus encompass the use of pharmaceutical compositions of the invention. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. For example unit dose container may be such that PRMT4 inhibitor solution is contained in a crushable sealed ampoule which in turn is enclosed in protective covering on which pressure is applied to crush the ampoule which then releases PRMT4 inhibitor solution for percolation through a flint-type tip which capped the ampoule in protective covering. When such packaging configuration is employed, care is taken to leave as little as possible or ideally no headspace in ampoule for any volatile portion of the solution to escape and cause a change in solution composition over a period of shelf life.

Although the description of pharmaceutical compositions provided herein are principally directed to pharmaceutical compositions which are suitable for ethical administration to humans, it will be understood by the skilled artisan that such compositions are generally suitable for administration to animals of all sorts, including mammals. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, vaginal, topical, intranasal, buccal, or another route of administration.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Non-limiting examples of such an additional pharmaceutically active agents are fluorouracil cream, imiquimod cream, ingenol mebutate gel, diclofenac sodium gel, topical retinoids, and tirbanibulin (Klisyri) ointment.

Controlled- or sustained-release formulations of a pharmaceutical composition of the invention may be made using conventional technology.

Formulations of a pharmaceutical composition suitable for topical administration comprise the active ingredient combined with a pharmaceutically acceptable carrier, such as sterile water or sterile isotonic saline. Formulations may be prepared, packaged, or sold in unit dosage form, such as in ampules, crushable or otherwise, or in multi-dose containers containing a preservative. Formulations for topical administration include, but are not limited to, suspensions, solutions, emulsions in oily or aqueous vehicles, solutions, suspensions, creams, pastes, oils, lotions, gels, foam, hydrogel, ointment, liposomes, emulsions, liquid crystal emulsions, nanoemulsions, implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile aqueous or oily suspension or solution. This suspension or solution may be formulated according to the known art, and may comprise, in addition to the active ingredient, additional ingredients such as the dispersing agents, wetting agents, or suspending agents described herein. Such sterile formulations may be prepared using a non-toxic acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other formulations that are useful include those which comprise the active ingredient in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

In some embodiments, the pharmaceutical compositions of the invention may be contained in a crushable ampule irrespective of the route of delivery to the patient.

It is contemplated that any embodiment discussed in this specification may be implemented with respect to any method or composition of the invention, and vice versa. Furthermore, compositions of the invention may be used to achieve methods of the invention.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

Dosing Regimes. The present methods for treating AD or pre-AD are carried out by administering a therapeutic for a time and in an amount sufficient to result in decreased memory problems or other symptoms of AD.

The amount and frequency of administration of the compositions can vary depending on, for example, what is being administered, the state of the patient, and the manner of administration. In therapeutic applications, compositions can be administered to a patient suffering from AD or pre-AD in an amount sufficient to relieve or least partially relieve the symptoms of the AD or pre-AD and its complications. The dosage is likely to depend on such variables as the type and extent of progression of the AD or pre-AD, the severity of the AD or pre-AD, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the AD or pre-AD or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol). Administration to humans is preferably in a dosage of between 0.1 mg/kg and 10.0 mg/kg, more preferably between 0.5 mg/kg and 5.0 mg/kg, and most preferably between 1.0 and 3.0 mg/kg.

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits. Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the AD or pre-AD.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including.” “comprising” or “having” and variations thereof herein is meant to encompass the items listed thereafter and equivalents thereof as well as additional items, while only the terms “consisting of” and “consisting only of” are to be construed in the limitative sense.

Wherefore, I/we claim:

1. A method of treating Alzheimer's disease or a pre-Alzheimer's disease condition in a patient comprising:
administering a pharmaceutical composition containing a therapeutically effective dose therapeutic;
wherein the therapeutic contains a PRMT4 inhibitor, or a pharmaceutically acceptable salt, solvate, ester, amide, clathrate, stereoisomer, enantiomer, prodrug or analog thereof.

2. The method of claim 1, wherein the PRMT4 inhibitor is one of TP-064 and SCF^{FBXO9}.

3. The method of claim 2 wherein the PRMT4 inhibitor is TP-064.

4. The method of claim 2 wherein the PRMT4 inhibitor is SCF^{FBXO9}.

5. The method of claim 1, wherein the PRMT4 inhibitor is administered in a dosage of between 0.1 mg/kg and 30.0 mg/kg, with the dosage expressed in a ratio of mass of PRMT4 inhibitor to mass of patient.

6. The method of claim 5, wherein the dosage is between 1.0 mg/kg and 15.0 mg/kg.

7. The method of claim 5, wherein the dosage is between 7.5 mg/kg and 10.0 mg/kg.

8. The method of claim 1, wherein the pharmaceutical composition is administered for at least 7 days.

9. The method of claim 1, wherein the patient does not display memory loss symptomatic of Alzheimer's disease.

10. The method of claim 1, wherein the patient displays memory loss symptomatic of Alzheimer's disease.

11. The method of claim 1, wherein the pharmaceutical composition is administered in an aerosol form.

12. The method of claim 11, wherein the pharmaceutical composition is administered with an inhaler device.
